SEQUENCE LISTING (79 sequences)

SEQ ID NO: 1 - protein, 110 aa, Homo sapiens
ETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 2 - protein, 110 aa, Homo sapiens
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 3 - protein, 315 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 4 - protein, 315 aa, Homo sapiens
GNCWLRQAKNGRCQVLYKTELSKEECCSTGRLSTSWTEEDVNDNTLFKWMIFNGGAPNCIPCKETCENVDCGPGQSCVVDQTGSPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 5 - protein, 288 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 6 - protein, 110 aa, Artificial
  Modified human ActRIIB ECD
ETRECIYYNANWELERTNQSGLERCEGDQDKRLHCYASWRNSSGTIELVKKGCWLDDINCYDRQECVATKENPQVYFCCCEGNFCNERFTHLPEAGGPEVTYEPPPTAPT

SEQ ID NO: 7 - protein, 110 aa, Artificial
  Modified human ActRIIB ECD
ETRECIYYNANWELERTNQSGLERCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 8 - protein, 107 aa, Artificial
  Modified human ActRIIB ECD
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 9 - protein, 109 aa, Artificial
  Modified human ActRIIA ECD
GAILGRSETQECLFYNANWELERTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 10 - protein, 452 aa, Artificial
  Anti-Activin antibody heavy chain amino acid sequence
QVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNVYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 213 aa, Artificial
  Anti-Activin A antibody light chain amino acid sequence
SYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 122 aa, Artificial
  Anti-Activin A antibody heavy chain variable region amino acid sequence
QVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSS

SEQ ID NO: 13 - protein, 106 aa, Artificial
  Anti-Activin A antibody light chain variable region amino acid sequence
SYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVL

SEQ ID NO: 14 - protein, 453 aa, Artificial
  Anti-Activin A antibody heavy chain amino acid sequence
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 215 aa, Artificial
  Anti-Activin A antibody light chain amino acid sequence
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 126 aa, Artificial
  Anti-Activin A antibody heavy chain variable region amino acid sequence
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSS

SEQ ID NO: 17 - protein, 108 aa, Artificial
  Anti-Activin A antibody light chain variable region amino acid sequence
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 18 - protein, 137 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 19 - protein, 162 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 20 - protein, 447 aa, Artificial
  Anti-TGF-Beta antibody heavy chain amino acid sequence
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 215 aa, Artificial
  Anti-TGF-Beta antibody light chain amino acid sequence
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 120 aa, Artificial
  Anti-TGF-Beta antibody heavy chain variable region amino acid sequence
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 23 - protein, 108 aa, Artificial
  Anti-TGF-Beta antibody light chain variable region amino acid sequence
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 24 - protein, 447 aa, Artificial
  Anti-RANKL antibody heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 215 aa, Artificial
  Anti-RANKL antibody light chain amino acid sequence
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 122 aa, Artificial
  Anti-RANKL antibody heavy chain variable region amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 27 - protein, 108 aa, Artificial
  Anti-RANKL antibody light chain variable region amino acid sequence
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 28 - protein, 401 aa, Homo sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 29 - protein, 571 aa, Artificial
  A112 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 30 - protein, 571 aa, Artificial
  A114 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 31 - protein, 776 aa, Artificial
  A231 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 32 - protein, 776 aa, Artificial
  A113 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSGNCWLRQAKNGRCQVLYKTELSKEECCSTGRLSTSWTEEDVNDNTLFKWMIFNGGAPNCIPCKETCENVDCGPGQSCVVDQTGSPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 33 - protein, 749 aa, Artificial
  A232 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 34 - protein, 571 aa, Artificial
  A233 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSETRECIYYNANWELERTNQSGLERCEGDQDKRLHCYASWRNSSGTIELVKKGCWLDDINCYDRQECVATKENPQVYFCCCEGNFCNERFTHLPEAGGPEVTYEPPPTAPT

SEQ ID NO: 35 - protein, 571 aa, Artificial; identical to SEQ ID NO: 34
  A234 Heavy chain amino acid sequence

SEQ ID NO: 36 - protein, 568 aa, Artificial
  A235 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 37 - protein, 570 aa, Artificial
  A236 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSGAILGRSETQECLFYNANWELERTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 38 - protein, 598 aa, Artificial
  A239 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 39 - protein, 623 aa, Artificial
  A240 Heavy chain amino acid sequence
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGSGGGGSGGGGSTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 40 - protein, 736 aa, Artificial
  A242 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 41 - protein, 736 aa, Artificial
  A243 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 42 - protein, 941 aa, Artificial
  A244 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 43 - protein, 941 aa, Artificial
  A245 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSGNCWLRQAKNGRCQVLYKTELSKEECCSTGRLSTSWTEEDVNDNTLFKWMIFNGGAPNCIPCKETCENVDCGPGQSCVVDQTGSPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 44 - protein, 914 aa, Artificial
  A246 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 45 - protein, 736 aa, Artificial
  A247 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSETRECIYYNANWELERTNQSGLERCEGDQDKRLHCYASWRNSSGTIELVKKGCWLDDINCYDRQECVATKENPQVYFCCCEGNFCNERFTHLPEAGGPEVTYEPPPTAPT

SEQ ID NO: 46 - protein, 736 aa, Artificial
  A248 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSETRECIYYNANWELERTNQSGLERCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 47 - protein, 733 aa, Artificial
  A249 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 48 - protein, 735 aa, Artificial
  A250 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSGAILGRSETQECLFYNANWELERTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 49 - protein, 846 aa, Artificial
  A251 Heavy chain amino acid sequence
QVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNVYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 50 - protein, 847 aa, Artificial
  A252 Heavy chain amino acid sequence
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGSGGGGSGGGGSETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 51 - protein, 763 aa, Artificial
  A253 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 52 - protein, 788 aa, Artificial
  A254 amino acid sequence
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 53 - protein, 841 aa, Artificial
  A255 Heavy chain amino acid sequence
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGSGGGGSGGGGSETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 54 - protein, 585 aa, Artificial
  A237 Heavy chain amino acid sequence
QVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSSGGGGSGGGGSGGGGSEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 55 - protein, 336 aa, Artificial
  A237 Light chain amino acid sequence
SYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVLGGGGSGGGGSGGGGSEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 589 aa, Artificial
  A238 Heavy chain amino acid sequence
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSSGGGGSGGGGSGGGGSEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - protein, 338 aa, Artificial
  A238 Light chain amino acid sequence
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 583 aa, Artificial
  A241 Heavy chain amino acid sequence
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSGGGGSGGGGSGGGGSEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 338 aa, Artificial
  A241 Light chain amino acid sequence
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKGGGGSGGGGSGGGGSEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 12 aa, Artificial
  Peptide linker sequence
GGGSGGGSGGGS

SEQ ID NO: 61 - protein, 4 aa, Artificial
  Peptide linker sequence
GGGS

SEQ ID NO: 62 - protein, 13 aa, Artificial
  Peptide linker sequence
GSSGGSGGSGGSG

SEQ ID NO: 63 - protein, 5 aa, Artificial
  Peptide linker sequence
GSSGT

SEQ ID NO: 64 - protein, 14 aa, Artificial
  Peptide linker sequence
GGGGSGGGGSGGGS

SEQ ID NO: 65 - protein, 17 aa, Artificial
  Peptide linker sequence
AEAAAKEAAAKEAAAKA

SEQ ID NO: 66 - protein, 20 aa, Artificial
  Peptide linker sequence
GGGGSGGGGSGGGGSGGGGS

SEQ ID NO: 67 - protein, 8 aa, Artificial
  Peptide linker sequence
GGGSGGGS

SEQ ID NO: 68 - protein, 5 aa, Artificial
  Peptide linker sequence
GSGST

SEQ ID NO: 69 - protein, 4 aa, Artificial
  Peptide linker sequence
GGSS

SEQ ID NO: 70 - protein, 5 aa, Artificial
  Peptide linker sequence
GGGGS

SEQ ID NO: 71 - protein, 4 aa, Artificial
  Peptide linker sequence
GGSG

SEQ ID NO: 72 - protein, 4 aa, Artificial
  Peptide linker sequence
SGGG

SEQ ID NO: 73 - protein, 4 aa, Artificial
  Peptide linker sequence
GSGS

SEQ ID NO: 74 - protein, 6 aa, Artificial
  Peptide linker sequence
GSGSGS

SEQ ID NO: 75 - protein, 8 aa, Artificial
  Peptide linker sequence
GSGSGSGS

SEQ ID NO: 76 - protein, 10 aa, Artificial
  Peptide linker sequence
GSGSGSGSGS

SEQ ID NO: 77 - protein, 12 aa, Artificial
  Peptide linker sequence
GSGSGSGSGSGS

SEQ ID NO: 78 - protein, 10 aa, Artificial
  Peptide linker sequence
GGGGSGGGGS

SEQ ID NO: 79 - protein, 15 aa, Artificial
  Peptide linker sequence
GGGGSGGGGSGGGGS

(19) United States
(12) Patent Application Publication
(10) Pub. No.: US 2023/0391880 A1
(43) Pub. Date: Dec. 7, 2023

(54) BIFUNCTIONAL ANTAGONISTS OF ACTIVIN/TGF-BETA AND RANKL AND USES THEREOF

(71) Applicants: HQ Han, Westlake Village, CA (US); Xiaolan Zhou, Newbury Park, CA (US)

(72) Inventors: HQ Han, Westlake Village, CA (US); Xiaolan Zhou, Newbury Park, CA (US)

(21) Appl. No.: 18/034,708

(22) PCT Filed: Nov. 5, 2021
(86) PCT No.: PCT/US21/58181

§ 371 Date: Apr. 30, 2023

Related U.S. Application Data
(60) Provisional application No. 63/113,992, filed on Nov. 16, 2020.
(60) Provisional application No. 63/112,380, filed on Nov. 11, 2020.

(51) Int. Cl. C07K 16/28; C07K 16/22; C07K 14/71

(52) U.S. Cl. CPC C07K 16/2875; C07K 16/22; C07K 14/71; A61K 2039/505

(57) ABSTRACT

This invention disclosure provides novel bifunctional molecules comprising at least one RANKL binding molecule and at least one Activin or TGF-β binding molecule, which are capable of sequestering RANKL and Activin or TGF-β in parallel. Also provided are pharmaceutical compositions of such bifunctional antagonist molecules and their therapeutic uses to treat various bone disorders whose pathogenesis involves the activation of both RANKL-NFκB and Activin/TGFβ-Smad2/3 signaling pathways, such as bone metastasis, bone loss in cancer, bone fragility in neuromuscular diseases, osteogenesis imperfecta, fracture, osteopenia and osteoporosis.

### RELATED PATENT APPLICATIONS

This application claims benefit of U.S. Provisional Application No. 63/113,922, filed on Nov. 15, 2020, and U.S. Provisional Application No. 63/112,380, filed on Nov. 11, 2020, each incorporated in its entirety by reference herein.

### BACKGROUND ART

Multiple factors regulate bone homeostasis by influencing bone formation or resorption. Balance between bone formation and resorption is crucial for sustaining bone mass and bone mineral density. Loss of this balance may result in bone loss and manifestation of bone disorders.

The receptor activator of nuclear factor-kappa B ligand (RANKL) signaling pathway plays a central role in the regulation of bone mass by stimulating osteoclast activity and increasing bone resorption, even though RANKL has no direct influence on osteoblast activity and bone formation.

Besides RANKL signaling pathway, Activin/TGF-β signaling pathway can also powerfully regulate bone mass. Remarkably, activation of Activin/TGF-β signaling not only stimulates osteoclast activity and bone resorption but also suppresses osteoblast activity and bone formation. Thus, Activin/TGF-β signaling pathway plays a dual action to regulate both bone resorption and bone formation. Recent studies demonstrated that pharmacological inhibition of Activin/TGF-β signaling pathway in vivo profoundly decreased bone resorption and at the same time markedly increased bone formation.

Mounting evidence indicates that the activities of both RANKL and Activin/TGF-β signaling pathways are increased in various skeletal disorders, indicating their parallel contribution to pathogenesis of bone disorders.

Importantly, activation of either RANKL signaling pathway or Activin/TGF-β signaling pathway can strongly induce epithelial-mesenchymal transition (EMT), a remodeling process that is critical for metastasis. In fact, expression levels of both RANKL and Activin/TGF-β have been shown to be elevated in bone metastasis, suggesting that increased RANKL and Activin/TGF-β signaling activities act in parallel to drive pathogenesis and progression of bone metastatic diseases in cancer.

In addition, muscle mass plays an important role in maintaining healthy bone. It has been shown that in wasting disease states, such as cancer cachexia, age-related sarcopenia and neuromuscular diseases, muscle wasting is intimately correlated with bone loss. Recent studies suggest that increased RANKL signaling as well as increased Activin/TGF-β signaling can both trigger muscle loss. Thus, inhibiting RANKL and Activin/TGF-β can enhance muscle mass and thereby indirectly benefit bone health.

Therefore, it is clearly of importance to develop novel bifunctional inhibitors to inhibit both RANKL signaling and Activin/TGF-β signaling for the treatment of bone disorders. Such novel bifunctional antagonists may achieve better efficacy and better response rate for treating certain bone disorders, such as bone metastasis and bone fragility, by inhibiting two major disease pathways at the same time.

### DISCLOSURE OF THE INVENTION

In one aspect, the present invention provides novel polypeptide-based bifunctional antagonists designed to simultaneously neutralize RANKL signaling and Activin/TGF-β signaling in a potent manner. In various embodiments, the bifunctional antagonist molecule is designed as depicted in FIGS. 1-5.

In various embodiments, the bifunctional antagonist molecule is a bifunctional polypeptide comprising a first antigen-binding molecule which specifically binds RANKL (“RANKL-Binding Polypeptide”) and a second antigen-binding molecule which specifically binds to either Activin ligand (“Activin-Binding Polypeptide”) or TGF-β ligand (“TGF-β-Binding Polypeptide”), which are capable of sequestering RANKL and Activin or TGF-β in parallel. In various embodiments, the “RANKL-Binding Polypeptide”, is selected from any polypeptide that is capable binding RANKL, which includes, but is not limited to, an anti-RANKL antibody or fragment of anti-RANKL antibody, wild-type osteoprotegerin (OPG) as well as modified OPG, and phage display-derived polypeptide capable of binding and sequestering RANKL. In various embodiments, the “Activin-Binding Polypeptide” is selected from any polypeptide that is capable binding Activin (i.e., Activin A, Activin B or Activin AB) and/or Activin-related ligand (i.e., GDF8 or GDF11), which includes, but is not limited to, an anti-Activin antibody (including anti-Activin A antibody and anti-Activin B antibody), a fragment of anti-Activin antibody, wild-type Activin Type 2A Receptor (ActRIIA) or Activin Type 2B Receptor (ActRIIB) extracellular domains (ECDs), modified ActRIIA and ActRIIB extracellular domains, wild-type and modified native Activin-binding proteins such as follistatin, follistatin-like protein and propeptide, and a phage display-derived polypeptide targeting Activin or Activin-related ligand. In various embodiments, the “TGF-β-Binding Polypeptide” is selected from the group consisting of an anti-TGF-β antibody, a fragment of anti-TGF-β antibody, wild-type TGF-β type-2 receptors (including TGFβRIIA and TGFβRIIB) extracellular domains (ECDs), modified TGFβRIIA and TGFβRIIB extracellular domains, and a phage display-derived antagonistic polypeptide targeting TGF-β ligand.

In various embodiments, the bifunctional antagonist molecule comprises an isolated antibody, or antigen-binding fragment thereof, that specifically binds to RANKL, and an isolated antibody, or antigen-binding fragment thereof, that specifically binds to either Activin or Activin-related ligand or to TGF-β ligand. In various embodiments, the isolated antibody or antigen-binding fragment thereof is selected from the group consisting of monoclonal Abs (mAbs), polyclonal Abs, Ab fragments (e.g., Fab, Fab′, F(ab′)2, Fv, Fc, etc.), chimeric Abs, mini-Abs or domain Abs (dAbs), dual specific Abs, bispecific Abs, heteroconjugate Abs, single chain Abs (SCA), single chain variable region fragments (ScFv), humanized Abs, fully human Abs, and any other modified configuration of the immunoglobulin (Ig) molecule that comprises an antigen recognition site of the required specificity. In various embodiments, the bifunctional molecule comprises an isolated antibody or antigen-binding fragment thereof selected from the group consisting of a fully human, humanized and chimeric antibody.

In various embodiments, the second antigen-binding molecule specifically binds an Activin or Activin-related ligand comprising an amino acid sequence set forth in SEQ ID NO: 1. In various embodiments, the second antigen-binding molecule specifically binds an Activin or Activin-related ligand comprising an amino acid sequence set forth in SEQ ID NO: 2. In various embodiments, the second antigen-binding molecule specifically binds an Activin or Activin-related ligand comprising an amino acid sequence set forth in SEQ ID NO: 3. In various embodiments, the second antigen-binding molecule specifically binds an Activin or Activin-related ligand comprising an amino acid sequence set forth in SEQ ID NO: 4. In various embodiments, the second antigen-binding molecule specifically binds an Activin or Activin-related ligand comprising an amino acid sequence set forth in SEQ ID NO: 5. In various embodiments, the second antigen-binding molecule specifically binds an Activin or Activin-related ligand comprising an amino acid sequence set forth in SEQ ID NO: 6. In various embodiments, the second antigen-binding molecule specifically binds an Activin or Activin-related ligand comprising an amino acid sequence set forth in SEQ ID NO: 7. In various embodiments, the second antigen-binding molecule specifically binds an Activin or Activin-related ligand comprising an amino acid sequence set forth in SEQ ID NO: 8. In various embodiments, the second antigen-binding molecule specifically binds an Activin or Activin-related ligand comprising an amino acid sequence set forth in SEQ ID NO: 9.

In various embodiments, the second antigen-binding molecule that specifically binds to Activin or Activin-related ligand is an isolated antibody selected from the group consisting of an antibody comprising the heavy chain amino acid sequence set forth in SEQ ID NO: 10; an antibody comprising the light chain amino acid sequence set forth in SEQ ID NO: 11; an antibody comprising the heavy chain amino acid sequence set forth in SEQ ID NO: 10 and the light chain amino acid sequence set forth in SEQ ID NO: 11; an antibody comprising the heavy chain variable region amino acid sequence set forth in SEQ ID NO: 12; an antibody comprising the light chain variable region amino acid sequence set forth in SEQ ID NO: 13; and an antibody comprising the heavy chain variable region amino acid sequence set forth in SEQ ID NO: 12 and the light chain variable region amino acid sequence set forth in SEQ ID NO: 13.

In various embodiments, the second antigen-binding molecule that specifically binds to Activin or Activin-related ligand is an isolated antibody selected from the group consisting of an antibody comprising the heavy chain amino acid sequence set forth in SEQ ID NO: 14; an antibody comprising the light chain amino acid sequence set forth in SEQ ID NO: 15; an antibody comprising the heavy chain amino acid sequence set forth in SEQ ID NO: 14 and the light chain amino acid sequence set forth in SEQ ID NO: 15; an antibody comprising the heavy chain variable region amino acid sequence set forth in SEQ ID NO: 16; an antibody comprising the light chain variable region amino acid sequence set forth in SEQ ID NO: 17; and an antibody comprising the heavy chain variable region amino acid sequence set forth in SEQ ID NO: 16 and the light chain variable region amino acid sequence set forth in SEQ ID NO: 17.

In various embodiments, the second antigen-binding molecule specifically binds a TGF-β ligand comprising an amino acid sequence set forth in SEQ ID NO: 18. In various embodiments, the second antigen-binding molecule specifically binds a TGF-β ligand comprising an amino acid sequence set forth in SEQ ID NO: 19.

In various embodiments, the second antigen-binding molecule that specifically binds to TGF-β ligand is an isolated antibody selected from the group consisting of an antibody comprising the heavy chain amino acid sequence set forth in SEQ ID NO: 20; an antibody comprising the light chain amino acid sequence set forth in SEQ ID NO: 21; an antibody comprising the heavy chain amino acid sequence set forth in SEQ ID NO: 20 and the light chain amino acid sequence set forth in SEQ ID NO: 21; an antibody comprising the heavy chain variable region amino acid sequence set forth in SEQ ID NO: 22; an antibody comprising the light chain variable region amino acid sequence set forth in SEQ ID NO: 23; and an antibody comprising the heavy chain variable region amino acid sequence set forth in SEQ ID NO: 22 and the light chain variable region amino acid sequence set forth in SEQ ID NO: 23.

In various embodiments, the bifunctional antagonist is a polypeptide molecule comprising a second antigen-binding molecule that specifically binds to Activin ligand or to TGF-β ligand, wherein the Activin ligand binding molecule is selected from the group of polypeptides comprising the amino acid sequence set forth in SEQ ID NOs: 1-17, and the TGF-β ligand binding molecule is selected from the group of polypeptides comprising the amino acid sequence set forth in SEQ ID NOs: 18-23.

In various embodiments, the RANKL-Binding Polypeptide is an isolated antibody selected from the group consisting of an antibody comprising the heavy chain amino acid sequence set forth in SEQ ID NO: 24; an antibody comprising the light chain amino acid sequence set forth in SEQ ID NO: 25; an antibody comprising the heavy chain amino acid sequence set forth in SEQ ID NO: 24 and the light chain amino acid sequence set forth in SEQ ID NO: 25; an antibody comprising the heavy chain variable region amino acid sequence set forth in SEQ ID NO: 26; an antibody comprising the light chain variable region amino acid sequence set forth in SEQ ID NO: 27; and an antibody comprising the heavy chain variable region amino acid sequence set forth in SEQ ID NO: 26 and the light chain variable region amino acid sequence set forth in SEQ ID NO: 27. In various embodiments, the RANKL-Binding Polypeptide is osteoprotegerin (OPG) comprising the amino acid sequence set forth in SEQ ID NO: 28.

In various embodiments, the bifunctional antagonist is a polypeptide molecule comprising a first antigen-binding molecule that specifically binds to RANKL and a second antigen-binding molecule that specifically binds to Activin ligand, wherein the bifunctional molecule is selected from the group consisting of: a bifunctional molecule comprising a heavy chain selected from a heavy chain comprising the amino acid sequence set forth in SEQ ID NOs: 29-37 and a light chain selected from a light chain comprising the amino acid sequence set forth in SEQ ID NO: 25.

In various embodiments, the bifunctional antagonist is a polypeptide molecule comprising a first antigen-binding molecule that specifically binds to RANKL, wherein the first antigen-binding molecule is an isolated antibody comprising the heavy chain amino acid sequence set forth in SEQ ID NO: 24 and the light chain amino acid sequence set forth in SEQ ID NO: 25; and a second antigen-binding molecule that specifically binds to Activin ligand, wherein the second antigen-binding molecule is an isolated antibody comprising the heavy chain amino acid sequence set forth in SEQ ID NO: 10 and the light chain amino acid sequence set forth in SEQ ID NO: 11. In various embodiments, the bifunctional antagonist is a polypeptide molecule comprising a first antigen-binding molecule that specifically binds to RANKL, wherein the first antigen-binding molecule is an isolated antibody comprising the heavy chain amino acid sequence set forth in SEQ ID NO: 24 and the light chain amino acid sequence set forth in SEQ ID NO: 25; and a second antigen-binding molecule that specifically binds to Activin ligand, wherein the second antigen-binding molecule is an isolated antibody comprising the heavy chain amino acid sequence set forth in SEQ ID NO: 14 and the light chain amino acid sequence set forth in SEQ ID NO: 15.

In various embodiments, the bifunctional antagonist is a polypeptide molecule comprising a first antigen-binding molecule that specifically binds to RANKL and a second antigen-binding molecule that specifically binds to TGF-β ligand, wherein the bifunctional molecule is selected from the group consisting of: a bifunctional molecule comprising a heavy chain selected from a heavy chain comprising the amino acid sequence set forth in SEQ ID NOs: 38-39 and a light chain selected from a light chain comprising the amino acid sequence set forth in SEQ ID NO: 25.

In various embodiments, the bifunctional antagonist is a polypeptide molecule comprising a first antigen-binding molecule that specifically binds to RANKL, wherein the first antigen-binding molecule is an isolated antibody comprising the heavy chain amino acid sequence set forth in SEQ ID NO: 24 and the light chain amino acid sequence set forth in SEQ ID NO: 25; and a second antigen-binding molecule that specifically binds to TGF-β ligand, wherein the second antigen-binding molecule is an isolated antibody comprising the heavy chain amino acid sequence set forth in SEQ ID NO: 20 and the light chain amino acid sequence set forth in SEQ ID NO: 21.

In various embodiments, the bifunctional antagonist is a polypeptide molecule comprising a first antigen-binding molecule that specifically binds to OPG ligand and a second antigen-binding molecule that specifically binds to Activin ligand, wherein the bifunctional molecule is selected from the group consisting of a polypeptide molecule comprising the amino acid sequence set forth in any one of SEQ ID NOs: 40-48.

In various embodiments, the bifunctional antagonist is a polypeptide molecule comprising a first antigen-binding molecule that specifically binds to OPG ligand and a second antigen-binding molecule that specifically binds to Activin ligand, wherein the bifunctional molecule is selected from the group consisting of: a bifunctional molecule comprising a heavy chain comprising the amino acid sequence set forth in SEQ ID NO: 49 and a light chain comprising the amino acid sequence set forth in SEQ ID NO: 11 and a bifunctional molecule comprising a heavy chain comprising the amino acid sequence set forth in SEQ ID NO: 50 and a light chain comprising the amino acid sequence set forth in SEQ ID NO: 15.

In various embodiments, the bifunctional antagonist is a polypeptide molecule comprising a first antigen-binding molecule that specifically binds to OPG ligand and a second antigen-binding molecule that specifically binds to TGF-β ligand, wherein the bifunctional antagonist is selected from the group consisting of a polypeptide molecule comprising the amino acid sequence set forth in any one of SEQ ID NOs: 51-52.

In various embodiments, the bifunctional antagonist is a polypeptide molecule comprising a first antigen-binding molecule that specifically binds to OPG ligand and a second antigen-binding molecule that specifically binds to TGF-β ligand, wherein the bifunctional molecule comprises a heavy chain comprising the amino acid sequence set forth in SEQ ID NO: 53 and a light chain comprising the amino acid sequence set forth in SEQ ID NO: 21.

In another aspect, the present disclosure provides a pharmaceutical composition comprising the isolated bifunctional antagonist molecules in admixture with a pharmaceutically acceptable carrier.

In various embodiments, the novel bifunctional antagonists of the present invention have broad applications for the treatment of various disorders whose pathogenesis involves the activation of both RANKL-NFκB and Activin/TGFβ-Smad2/3 signaling pathways including, but not limited to bone metastasis and skeletal-related events in cancer, including breast cancer, multiple myeloma, prostate cancer, lung cancer, kidney cancer, lymphoma, thyroid cancer, and other malignancies; osteolytic neoplasm, such as Giant Cell Tumor of Bone (GCTB); bone fractures, such as hip fracture; osteoporosis; glucocorticoid therapy induced bone loss; androgen deprivation therapy induced bone loss; muscle wasting disorders with bone loss, such as cancer cachexia, sarcopenia, muscular dystrophy, spinal muscular atrophy; and osteogenesis imperfecta. In various embodiments, the disorder is selected from the group consisting of bone metastasis, bone loss in cancer, bone fragility in neuromuscular diseases, osteogenesis imperfecta, fracture, osteopenia and osteoporosis.

In another aspect, the disclosure provides uses of the bifunctional antagonist molecules for making a medicament for the treatment of any disorder or condition as described herein.

In another aspect, the present disclosure provides isolated nucleic acid molecules comprising a polynucleotide encoding a bifunctional antagonist molecule of the present disclosure. In various embodiments, the isolated nucleic acid molecules comprise the polynucleotides described herein, and further comprise a polynucleotide encoding at least one heterologous protein described herein. In various embodiments, the nucleic acid molecules further comprise polynucleotides encoding the linkers or hinge linkers described herein.

In another aspect, the present disclosure provides vectors comprising the nucleic acids described herein. In various embodiments, the vector is an expression vector. In another aspect, the present disclosure provides isolated cells comprising the nucleic acids of the disclosure. In various embodiments, the cell is a host cell comprising the expression vector of the disclosure. In another aspect, methods of making the bifunctional antagonist molecules are provided by culturing the host cells under conditions promoting expression of the proteins or polypeptides.

In another aspect, provided is a method for producing a bifunctional antagonist molecule comprising a first antigen-binding molecule that specifically binds to RANKL and a second antigen-binding molecule that specifically binds to Activin/TGF-β as described herein, comprising the steps of a) transforming a host cell with vectors comprising polynucleotides encoding said bifunctional antagonist molecule, b) culturing the host cell according under conditions suitable for the expression of the bifunctional antagonist molecule and c) recovering the bifunctional antagonist molecule from the culture. The invention also encompasses a bifunctional antagonist molecule produced by the method of the invention.

### BRIEF DESCRIPTION OF THE FIGURES

FIG. 1 depicts a representative bifunctional antagonist molecule of the present invention capable of inhibiting RANKL and Activin or TGFβ through fusion between RANKL-binding polypeptide (fusion partner A) and Activin- or TGFβ-binding polypeptide (fusion partner B). The “RANKL-binding polypeptide”, as illustrated in this schematic, refers to any polypeptide that is capable binding RANKL, which includes, but not limited to, 1) anti-RANKL antibody or fragment of anti-RANKL antibody, 2) wild-type as well as modified osteoprotegerin (OPG), and 3) phage display-derived polypeptide capable of binding and sequestering RANKL. The “Activin- or TGFβ-binding polypeptide”, as illustrated in this schematic, refers to any polypeptide that is capable binding Activin (i.e., Activin A, Activin B or Activin AB), or TGFβ (i.e., TGFβ1, TGFβ2 or TGFβ3), which includes, but not limited to, 1) anti-Activin antibody or fragment of anti-Activin antibody and anti-TGFβ antibody or fragment of anti-TGFβ antibody, 2) extracellular domains (ECDs) of wild-type as well as modified Activin type-2 receptors (including ActRIIA and ActRIIB) and extracellular domains (ECDs) of wild-type or modified TGFβ type-2 receptors (including TGFβRIIA and TGFβRIIB), 3) Wild-type and modified follistatin proteins that bind and neutralize activin, and 4) phage display-derived antagonistic polypeptides capable of binding and neutralizing Activin or TGFβ. The “Linker”, as shown in this schematic, refers to various methods for fusing different polypeptide fusion partners to generate bispecific and multi-specific molecules, which includes, but not limited to, the use of any peptide linker or chemical linker.

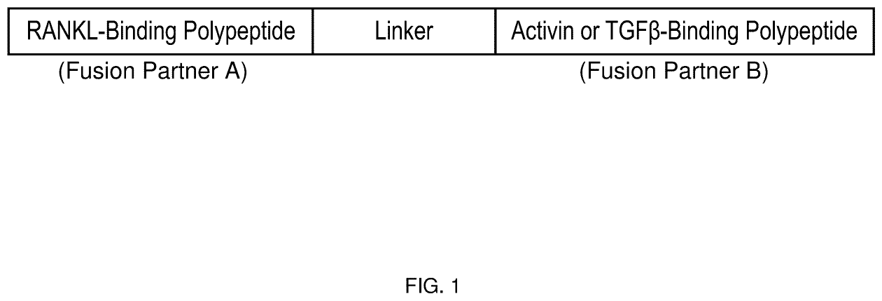

FIGS. 2A and 2B depict two representative bifunctional antagonist molecules of the present invention wherein: (A) the RANKL-binding polypeptide is an anti-RANKL antibody and the Activin-binding polypeptide is an Activin Receptor ECD attached via a linker to the heavy chain CH3 of the anti-RANKL antibody; or (B) the RANKL-binding polypeptide is an anti-RANKL antibody and the TGF-β-binding polypeptide is a TGF-β. Receptor ECD attached via a linker to the heavy chain CH3 of the anti-RANKL antibody. In alternative embodiments, the Activin Receptor ECD (or TGF-β Receptor ECD) is attached to the anti-Activin antibody (or anti-TGF-β antibody) via a linker at the heavy chain variable region (VH) of the antibody. In alternative embodiments, the Activin Receptor ECD (or TGF-β Receptor ECD) is attached to the anti-Activin antibody (or anti-TGF-β antibody) via a linker at the light chain variable region (VL) of the antibody. In alternative embodiments, the Activin Receptor ECD (or TGF-β Receptor ECD) is attached to the anti-Activin antibody (or anti-TGF-β antibody) via a linker at an internal site rather than at the heavy chain CH3, VL, or VH sites of the antibody.

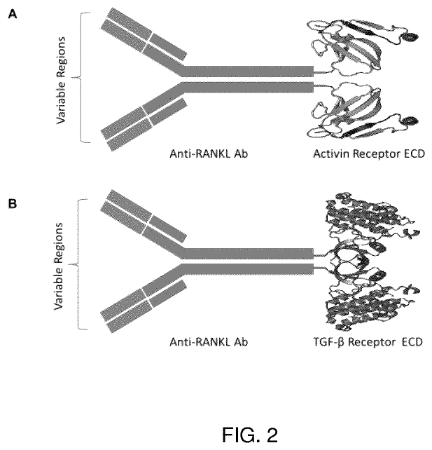

FIGS. 3A and 3B depict two representative bifunctional antagonist molecules of the present invention wherein: (A) the Activin-binding polypeptide is an anti-Activin antibody and the RANKL-binding polypeptide is OPG attached via a linker to the heavy chain CH3 of the anti-Activin antibody; or (B) the TGF-β-binding polypeptide is an anti-TGF-β antibody and the RANKL-binding polypeptide is a OPG attached via a linker to the heavy chain CH3 of the anti-TGF-β antibody. In alternative embodiments, the OPG is attached to the anti-Activin antibody (or anti-TGF-β antibody) via a linker at the heavy chain variable region (VH) of the antibody. In alternative embodiments, the OPG is attached to the anti-Activin antibody (or anti-TGF-β antibody) via a linker at the light chain variable region (VL) of the antibody. In alternative embodiments, the OPG is attached to the anti-Activin antibody (or anti-TGF-β antibody) via a linker at an internal site rather than at the heavy chain CH3, VL, or VH sites of the antibody.

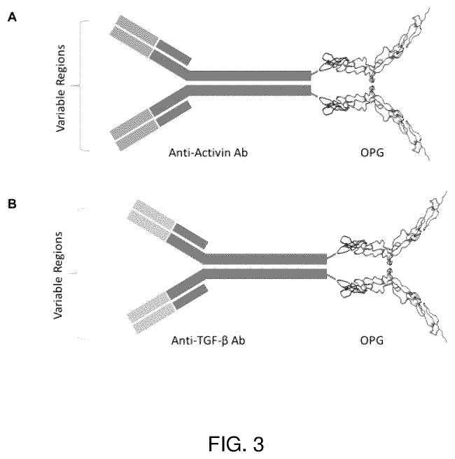

FIGS. 4A and 4B depict two representative bifunctional antagonist molecules of the present invention wherein: (A) the RANKL-binding polypeptide is OPG and the Activin-binding polypeptide is an Activin Receptor ECD attached via an Fc domain; or (B) the RANKL-binding polypeptide is OPG and the TGF-β-binding polypeptide is an TGF-β Receptor ECD attached via an Fc domain.

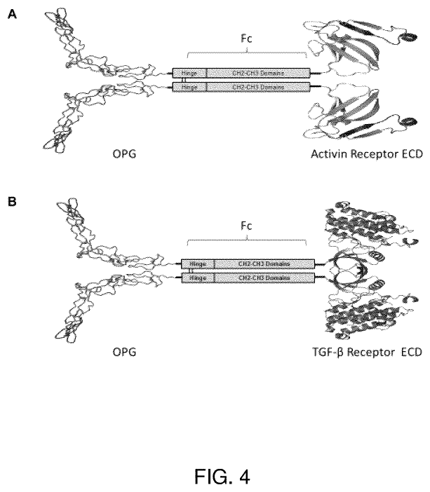

FIGS. 5A and 5B depict two representative bifunctional antagonist molecules of the present invention in the form of a bispecific antibody, wherein the bispecific antibody comprises (A) variable regions (VH and VL) of an anti-activin A antibody and the variable regions (VH and VL) of an anti-RANKL antibody, or (B) variable regions (VH and VL) of an anti-TGF-β antibody and variable regions (VH and VL) of an anti-RANKL antibody. Note that although the bispecific antibody examples illustrated in FIG. 5 are shown in only one specific configuration, bispecific antibodies comprising variable regions derived from both anti-activin A antibody and anti-anti-RANKL antibody or from both anti-TGF-β and anti-RANKL antibody can be constructed in a wide variety of configurations by those that are skilled in the art.

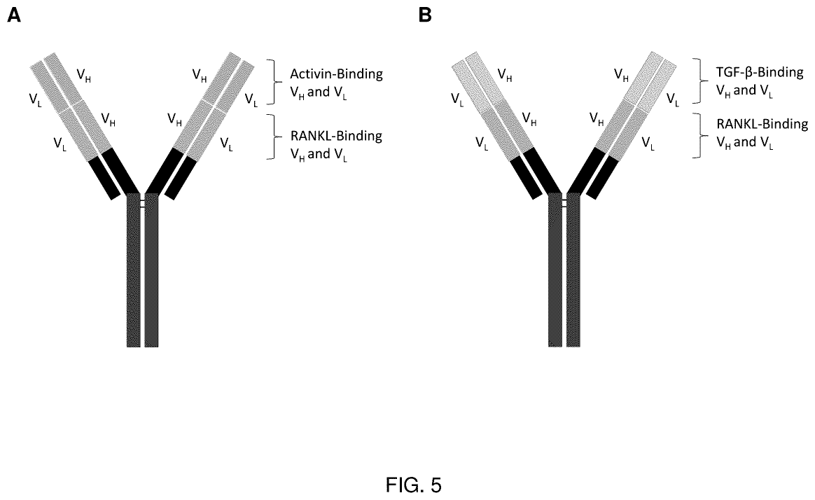

FIG. 6 depicts line graphs showing that bifunctional antagonist molecule A112 potently neutralizes Activin A, Activin B, Activin AB and Myostatin in cell-based assays. The IC₅₀ values were calculated and plotted using Prism software (GraphPad Software).

FIG. 7 depicts the photographs of RAW 264.7 cells showing the induction of osteoclastogenesis in response to RANKL and activin A and the ability of bifunctional antagonist A112 in compararion to anti-RANKL antibody or ActRIIA-Fc to inhibit the RANKL- and activin A-induced osteoclast formation.

FIG. 8 depicts bar graphs on osteoclast counts in RAW 264.7 cell cultures showing that in the presence RANKL and TGF-β1, bifunctional antagonist A112 was more effective than anti-RANKL antibody or ActRIIA-Fc in suppressing osteoclast formation.

FIG. 9 depicts the photographs of RAW 264.7 cell cultures showing the induction of osteoclast formation in response to RANKL and TGF-β1 and the effect of bifunctional antagonist A240 in compararion to anti-RANKL antibody or TGFRII-Fc in inhibiting the RANKL- and activin A-induced osteoclast formation.

FIG. 10 depicts bar graphs on osteoclast counts in RAW 264.7 cells showing that in the presence RANKL and TGF-β1, bifunctional antagonist A240 was more effective than anti-RANKL antibody or TGFRII-Fc in preventing osteoclast formation.

FIG. 11 depicts the representative microCT images of bone volume of the distal femur in the different mouse groups showing a marked bone loss resulting from dexamethasone and a complete prevention of the bone loss by the combination treatment with ActRIIA-Fc and anti-murine RANKL antibody.

FIG. 12 depicts the quantitative bar graphs of microCT imaging data on trabecular bone thickness of the distal femur in different mouse groups as illustrated in the figure. Note that the combination treatment with ActRIIA-Fc and anti-murine RANKL antibody prevented glucocorticoid-induced bone loss more effectively compared to treatment with ActRIIA-Fc alone or anti-murine RANKL antibody alone.

### MODE(S) FOR CARRYING OUT THE DISCLOSURE

### Definitions

The terms “polypeptide”, “peptide” and “protein” are used interchangeably herein to refer to a polymer of amino acid residues. In various embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties.

An amino acid “substitution” as used herein refers to the replacement in a polypeptide of one amino acid at a particular position in a parent polypeptide sequence with a different amino acid. Amino acid substitutions can be generated using genetic or chemical methods well known in the art. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:
1) Alanine (A), Serine (S), and Threonine (T)
2) Aspartic acid (D) and Glutamic acid (E)
3) Asparagine (N) and Glutamine (Q)
4) Arginine (R) and Lysine (K)
5) Isoleucine (I), Leucine (L), Methionine (M), and Valine (V)
6) Phenylalanine (F), Tyrosine (Y), and Tryptophan (W)

A “non-conservative amino acid substitution” refers to the substitution of a member of one of these classes for a member from another class. In making such changes, according to various embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in various embodiments, the substitution of amino acids whose hydropathic indices are within ±2 is included. In various embodiments, those that are within ±1 are included, and in various embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In various embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.+−.1); glutamate (+3.0.+−.1); serine (+0.3); asparagine (+0.2); glutamine (+0.2); glycine (0); threonine (−0.4); proline (−0.5.+−.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in various embodiments, the substitution of amino acids whose hydrophilicity values are within ±2 is included, in various embodiments, those that are within ±1 are included, and in various embodiments, those within ±0.5 are included. Exemplary amino acid substitutions are set forth in Table 1.

TABLE 1

| Original Residues | Exemplary Substitutions | Preferred Substitutions |
|---|---|---|
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | |
| Trp | Tyr, Phe | Tyr |
| Tyr | Trp, Phe, Thr, Ser | Phe |
| Val | Ile, Met, Leu, Phe, | Leu |

A skilled artisan will be able to determine suitable variants of polypeptides as set forth herein using well-known techniques. In various embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a polypeptide that correspond to amino acid residues important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three-dimensional structure. In various embodiments, one skilled in the art may choose to not make radical changes to amino acid residues predicted to be on the surface of the polypeptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “polypeptide fragment” and “truncated polypeptide” as used herein refers to a polypeptide that has an amino-terminal and/or carboxy-terminal deletion as compared to a corresponding full-length protein. In various embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant”, “hybrid polypeptide” and “polypeptide mutant” as used herein refers to a polypeptide that comprises an amino acid sequence wherein one or more amino acid residues are inserted into, deleted from and/or substituted into the amino acid sequence relative to another polypeptide sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Hybrids of the present disclosure include fusion proteins.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 95%, or at least 99% or more sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 95%, or at least 99% or more sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., J. Mol. Biol. 215:403-10, 1990 (with special reference to the published default setting, i.e., parameters w=4, t=17) and Altschul et al., Nucleic Acids Res., 25:3389-3402, 1997. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix.

In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The term “modification” as used herein refers to any manipulation of the peptide backbone (e.g., amino acid sequence) or the post-translational modifications (e.g., glycosylation) of a polypeptide.

The term “antigen binding molecule” as used herein refers in its broadest sense to a molecule that specifically binds an antigenic determinant. Examples of antigen binding molecules are antibodies, antibody fragments and scaffold antigen binding proteins. An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

As used herein, the term “antigen-binding site” refers to the part of the antigen binding molecule that specifically binds to an antigenic determinant. More particularly, the term “antigen-binding site” refers the part of an antibody that comprises the area which specifically binds to and is complementary to part or all of an antigen. Where an antigen is large, an antigen binding molecule may only bind to a particular part of the antigen, which part is termed an epitope. An antigen-binding site may be provided by, for example, one or more variable domains (also called variable regions). Preferably, an antigen-binding site comprises an antibody light chain variable region (VL) and an antibody heavy chain variable region (VH).

As used herein, the term “antigenic determinant” is synonymous with “antigen” and “epitope,” and refers to a site (e.g., a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g., humans) and rodents (e.g., mice and rats), unless otherwise indicated. In various embodiments, the antigen is a human protein.

The term “antibody” herein is used in the broadest sense and encompasses various antibody structures, including but not limited to monoclonal antibodies, polyclonal antibodies, monospecific and bifunctional antibodies (e.g., bispecific antibodies), and antibody fragments so long as they exhibit the desired antigen-binding activity.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antibody is able to specifically bind to at least two distinct antigenic determinants, for example two binding sites each formed by a pair of an antibody heavy chain variable domain (VH) and an antibody light chain variable domain (VL) binding to different antigens or to different epitopes on the same antigen. Such a bispecific antibody is an 1+1 format. Other bispecific antibody formats are 2+1 formats (comprising two binding sites for a first antigen or epitope and one binding site for a second antigen or epitope) or 2+2 formats (comprising two binding sites for a first antigen or epitope and two binding sites for a second antigen or epitope). Typically, a bispecific antibody comprises two antigen binding sites, each of which is specific for a different antigenic determinant.

The term “valent” as used within the current application denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule. The bispecific antibodies according to the invention are at least “bivalent” and may be “trivalent” or “multivalent” (e.g., “tetravalent” or “hexavalent”). In various embodiments, the antibodies of the present invention have two or more binding sites and are bispecific. That is, the antibodies may be bispecific even in cases where there are more than two binding sites (i.e. that the antibody is trivalent or multivalent). In particular, the invention relates to bispecific bivalent antibodies, having one binding site for each antigen they specifically bind to.

The terms “full-length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called alpha (IgA), delta (IgD), epsilon (IgE), gamma (IgG), or mu (IgM), some of which may be further divided into subtypes, e.g., gamma 1 (IgG1), gamma 2 (IgG2), gamma 3 (IgG3), gamma 4 (IgG4), alpha 1 (IgA1) and alpha 2 (IgA2). The light chain of an antibody may be assigned to one of two types, called kappa and lambda, based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g., scFv); bifunctional antibodies formed from antibody fragments and single domain antibodies. For a review of certain antibody fragments, see Hudson et al., Nat Med 9, 129-134 (2003). For a review of scFv fragments, see e.g., Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g., U.S. Pat. No. 6,248,516 B1). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full-length antibodies. Antibody fragments can be made by various techniques, including but not limited to proteolytic digestion of an intact antibody as well as production by recombinant host cells (e.g., E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteines from the antibody hinge region. Fab′-SH are Fab′ fragments wherein the cysteine residue(s) of the constant domains bear a free thiol group. Pepsin treatment yields an F(ab′)₂ fragment that has two antigen-combining sites (two Fab fragments) and a part of the Fc region.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g., position 44 in the variable heavy chain and position 100 in the variable light chain according to Kabat numbering).

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g., described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding molecule and thereby providing the antigen binding property of full-length antibodies.

The term “Fc domain” or “Fc region” herein is used to define a C-terminal region of an antibody heavy chain that contains at least a portion of the constant region. The term includes native sequence Fc regions and variant Fc regions. Particularly, a human IgG heavy chain Fc region extends from Cys226, or from Pro230, to the carboxyl-terminus of the heavy chain. However, the C-terminal lysine (Lys447) of the Fc region may or may not be present. The amino acid sequences of the heavy chains are always presented with the C-terminal lysine, however variants without the C-terminal lysine are included in the invention.

An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g., a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “knob-into-hole” technology is described e.g., in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). The protuberance and cavity can be made by altering the nucleic acid encoding the polypeptides, e.g., by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

A “blocking” antibody or an “antagonist” antibody is one that inhibits or reduces a biological activity of the antigen it binds. In some embodiments, blocking antibodies or antagonist antibodies substantially or completely inhibit the biological activity of the antigen. For example, the bispecific antibodies of the invention block the signaling through RANKL so as to inhibit RANKL-NFκB signaling pathway and simultaneously block the signaling through TGF-β or Activin so as to inhibit TGF-β/Activin-Smad2/3 signaling pathway.

As used herein, “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g., Surface Plasmon Resonance (SPR) technique (analyzed on a BlAcore instrument) (Liljeblad et al., Glyco J 17, 323-329 (2000)), and traditional binding assays (Heeley, Endocr Res 28, 217-229 (2002)).

The terms “affinity” or “binding affinity” as used herein refers to the strength of the sum total of non-covalent interactions between a single binding site of a molecule (e.g., an antibody) and its binding partner (e.g., an antigen). The affinity of a molecule X for its partner Y can generally be represented by the dissociation constant (KD), which is the ratio of dissociation and association rate constants (koff and kon, respectively). A particular method for measuring affinity is Surface Plasmon Resonance (SPR). As used herein, the term “high affinity” of an antibody refers to an antibody having a Kd of 10⁻⁹ M or less and even more particularly 10⁻¹⁰ M or less for a target antigen. The term “low affinity” of an antibody refers to an antibody having a Kd of 10⁻⁸ M or higher. The term “reduced binding”, as used herein refers to a decrease in affinity for the respective interaction, as measured for example by SPR. Conversely, “increased binding” refers to an increase in binding affinity for the respective interaction.

The term “a bispecific antibody comprising a first antigen-binding molecule that specifically binds to RANKL and a second antigen-binding molecule that specifically binds to either Activin ligand or TGF-β ligand”, “a bispecific antibody that specifically binds RANKL and either Activin ligand or TGF-β ligand”, “bispecific antigen binding molecule specific for RANKL and either Activin ligand or TGF-β ligand” are used interchangeably herein and refer to a bispecific antibody that is capable of binding RANKL and either Activin ligand or TGF-β ligand with sufficient affinity such that the antibody is useful as a diagnostic and/or therapeutic agent in targeting RANKL and either Activin ligand and TGF-β ligand.

The terms “anti-RANKL antibody” and “an antibody comprising an antigen-binding site that binds to RANKL” refer to an antibody that is capable of binding RANKL, especially a RANKL polypeptide expressed on a cell surface, with sufficient affinity such that the antibody is useful as a diagnostic and/or therapeutic agent in targeting RANKL. In one embodiment, the extent of binding of an anti-RANKL antibody to an unrelated, non-RANKL protein is less than about 10% of the binding of the antibody to RANKL as measured, e.g., by radioimmunoassay (RIA) or flow cytometry (FACS) or by a Surface Plasmon Resonance assay using a biosensor system such as a Biacore® system. In certain embodiments, an antigen binding molecule that binds to human RANKL has a KD value of the binding affinity for binding to human RANKL of, e.g., from 10⁻⁸ M to 10⁻¹³ M. In one preferred embodiment the respective KD value of the binding affinities is determined in a Surface Plasmon Resonance assay using the Extracellular domain (ECD) of human RANKL (RANKL-ECD) for the RANKL binding affinity. The term “anti-RANKL antibody” also encompasses bispecific antibodies that are capable of binding RANKL and a second antigen.

The terms “anti-Activin antibody” and “an antibody comprising an antigen-binding site that binds to Activin” refer to an antibody that is capable of binding Activin, especially a Activin polypeptide expressed on a cell surface, with sufficient affinity such that the antibody is useful as a diagnostic and/or therapeutic agent in targeting Activin. In one embodiment, the extent of binding of an anti-Activin antibody to an unrelated, non-Activin protein is less than about 10% of the binding of the antibody to Activin as measured, e.g., by radioimmunoassay (RIA) or flow cytometry (FACS) or by a Surface Plasmon Resonance assay using a biosensor system such as a Biacore® system. In certain embodiments, an antigen binding molecule that binds to human Activin has a KD value of the binding affinity for binding to human Activin of, e.g., from 10⁻⁸ M to 10⁻¹³ M. In one preferred embodiment the respective KD value of the binding affinities is determined in a Surface Plasmon Resonance assay using the Extracellular domain (ECD) of human Activin (Activin-ECD) for the Activin binding affinity. The term “anti-Activin antibody” also encompasses bispecific antibodies that are capable of binding Activin and a second antigen.

The terms “anti-TGF-β antibody” and “an antibody comprising an antigen-binding site that binds to TGF-β” refer to an antibody that is capable of binding TGF-β, especially a TGF-β polypeptide expressed on a cell surface, with sufficient affinity such that the antibody is useful as a diagnostic and/or therapeutic agent in targeting TGF-β. In one embodiment, the extent of binding of an anti-TGF-β antibody to an unrelated, non-TGF-β protein is less than about 10% of the binding of the antibody to TGF-β as measured, e.g., by radioimmunoassay (RIA) or flow cytometry (FACS) or by a Surface Plasmon Resonance assay using a biosensor system such as a Biacore® system. In certain embodiments, an antigen binding molecule that binds to human TGF-β has a KD value of the binding affinity for binding to human TGF-β of, e.g., from 10⁻⁸ M to 10⁻¹³ M. In one preferred embodiment the respective KD value of the binding affinities is determined in a Surface Plasmon Resonance assay using the Extracellular domain (ECD) of human TGF-β (TGF-β-ECD) for the TGF-β binding affinity. The term “anti-TGF-β antibody” also encompasses bispecific antibodies that are capable of binding TGF-β and a second antigen.

The term “fusion protein” as used herein refers to a fusion polypeptide molecule comprising two or more genes that originally coded for separate proteins, wherein the components of the fusion protein are linked to each other by peptide-bonds, either directly or through peptide linkers. The term “fused” as used herein refers to components that are linked by peptide bonds, either directly or via one or more peptide linkers.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc.

The term “peptide linker” as used herein refers to a peptide comprising one or more amino acids, typically about 2-20 amino acids. Peptide linkers are known in the art or are described herein. Suitable, non-immunogenic linker peptides include, for example, (G₄S)ₙ, (SG₄)ₙ or G₄(SG₄)n peptide linkers. “n” is generally a number between 1 and 10, typically between 2 and 4.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

As used herein, “treatment” (and grammatical variations thereof such as “treat” or “treating”) refers to clinical intervention in an attempt to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

The phrase “administering” or “cause to be administered” refers to the actions taken by a medical professional (e.g., a physician), or a person controlling medical care of a patient, that control and/or permit the administration of the agent(s)/compound(s) at issue to the patient. Causing to be administered can involve diagnosis and/or determination of an appropriate therapeutic regimen, and/or prescribing particular agent(s)/compounds for a patient. Such prescribing can include, for example, drafting a prescription form, annotating a medical record, and the like. Where administration is described herein, “causing to be administered” is also contemplated.

The terms “patient,” “individual,” and “subject” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context. In various embodiments, the patient may be an immunocompromised patient or a patient with a weakened immune system including, but not limited to patients having primary immune deficiency, AIDS; cancer and transplant patients who are taking certain immunosuppressive drugs; and those with inherited diseases that affect the immune system (e.g., congenital agammaglobulinemia, congenital IgA deficiency). In various embodiments, the patient has an immunogenic cancer, including, but not limited to bladder cancer, lung cancer, melanoma, and other cancers reported to have a high rate of mutations (Lawrence et al., Nature, 499(7457): 214-218, 2013).

The term “immunotherapy” refers to cancer treatments which include, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, RANKL, OX-40, CD137, GITR, LAG3, TIM-3, SIRP, CD40, CD47, Siglec 8, Siglec 9, Siglec 15, TIGIT and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using Bacilli Calmette-Guerin (BCG); treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, immunotherapy alone, surgery alone, or combinations thereof. For ease of description and not limitation, it will be understood that the refractory tumor cells are interchangeable with resistant tumor.

The term “polymer” as used herein generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions.

“Hybridizing specifically to” or “specific hybridization” or “selectively hybridize to”, refers to the binding, duplexing, or hybridizing of a nucleic acid molecule preferentially to a particular nucleotide sequence under stringent conditions when that sequence is present in a complex mixture (e.g., total cellular) DNA or RNA. The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence-dependent and are different under different environmental parameters. An extensive guide to the hybridization of nucleic acids can be found in Tijssen, 1993, Laboratory Techniques in Biochemistry and Molecular Biology—Hybridization with Nucleic Acid Probes, part I, chapter 2, “Overview of principles of hybridization and the strategy of nucleic acid probe assays”, Elsevier, N.Y.; Sambrook et al., 2001, Molecular Cloning: A Laboratory Manual, Cold Spring Harbor Laboratory, 3.sup.rd ed., NY; and Ausubel et al., eds., Current Edition, Current Protocols in Molecular Biology, Greene Publishing Associates and Wiley Interscience, NY.

Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than about 100 complementary residues on a filter in a Southern or northern blot is 50% formalin with 1 mg of heparin at 42° C., with the hybridization being carried out overnight. An example of highly stringent wash conditions is 0.15 M NaCl at 72° C. for about 15 minutes. An example of stringent wash conditions is a 0.2× SSC wash at 65° C. for 15 minutes. See Sambrook et al. for a description of SSC buffer. A high stringency wash can be preceded by a low stringency wash to remove background probe signal. An exemplary medium stringency wash for a duplex of, e.g., more than about 100 nucleotides, is 1× SSC at 45° C. for 15 minutes. An exemplary low stringency wash for a duplex of, e.g., more than about 100 nucleotides, is 4-6× SSC at 40° C. for 15 minutes. In general, a signal to noise ratio of 2× (or higher) than that observed for an unrelated probe in the particular hybridization assay indicates detection of a specific hybridization.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe,” when used in reference to a polynucleotide, refers to a polynucleotide that is capable of specifically hybridizing to a designated sequence of another polynucleotide. A probe specifically hybridizes to a target complementary polynucleotide but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties. In instances where a probe provides a point of initiation for synthesis of a complementary polynucleotide, a probe can also be a primer.

A “vector” is a polynucleotide that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. A nucleotide sequence is “operably linked” to a regulatory sequence if the regulatory sequence affects the expression (e.g., the level, timing, or location of expression) of the nucleotide sequence.

A “host cell” is a cell that can be used to express a polynucleotide of the disclosure. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated molecule” (where the molecule is, for example, a polypeptide or a polynucleotide) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The terms “label” or “labeled” as used herein refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In various embodiments, labels are attached by spacer arms of various lengths to reduce potential steric hindrance.

The term “heterologous” as used herein refers to a composition or state that is not native or naturally found, for example, that may be achieved by replacing an existing natural composition or state with one that is derived from another source. Similarly, the expression of a protein in an organism other than the organism in which that protein is naturally expressed constitutes a heterologous expression system and a heterologous protein.

It is understood that aspect and embodiments of the disclosure described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variations.

### TGF-β Ligands and Activin Ligands

TGF-β (TGF-β1, TGF-β2 and TGF-β3) mediates Smad2/3 signaling through its binding and activation of the high-affinity receptors TGFβRII and TGFβRIIB on the cell surface. TGF-β plays a critical role in the regulation of a wide range of biology activities, including immune function, cell proliferation and differentiation, fibrogenesis, epithelial-mesenchymal transition, hematopoiesis, myogenesis and bone remodeling. Elevated TGF-β levels and consequently increased Smad2/3 signaling have been implicated in pathogenesis and progression of many disease conditions including cancer, fibrosis, anemia, bone metastasis, bone loss, pain, muscle loss, insulin resistance, chronic kidney disease, liver disease, and cardiovascular diseases.

In addition, as a subset of TGF-β superfamily, Activins and related proteins (including Activin A, Activin B, Activin AB, GDF-8 and GDF-11) also mediate Smad2/3 signaling through binding and activation of their high-affinity receptors ActRIIA and ActRIIB on the cell surface. Activin and related proteins regulate a wide range of biology activities, including immune function, cell differentiation, myogenesis, fibrogenesis, bone remodeling, hematopoiesis, and reproductive physiology. Follistatin (FST), a secreted glycoprotein, binds to the Activins and Activin-related ligands to negatively control their signaling activities. Overexpression of Activins and related ligands and increased Smad2/3 signaling have been implicated in pathogenesis and progression of many grievous conditions, such as cancer, fibrosis, anemia, bone metastasis, bone fragility, facture, muscle wasting disorders, cachexia, pulmonary hypertension, pain, insulin resistance, chronic kidney disease, liver disease, myocardial infarction and heart failure.

TGF-β/Activin-Smad2/3 signaling pathway plays a central role in the pathogenesis and progression of fibrosis. A key mechanism underlying pathogenesis and progression of fibrosis is the increased TGF-β/Activin-Smad2/3 signaling, which leads to proliferation and activation of fibroblasts and consequently, overexpression of extracellular matrix components such as COL1A1 , COL1A2, COL3A1, COL5A2, COL6A1 and COL6A3 in the disease tissue. Evidence indicates that TGF-β and Activin are both upregulated during fibrosis. When elevated, either TGF-β or Activin can cause fibroblast activation, leading to fibrosis. Since TGF-β and Activin are both elevated, it is important to inhibit not only TGF-β but also Activin in order to more effectively attenuate fibrosis.

Activins, including Activin A, Activin B and Activin AB, and Activin-related proteins, including Myostatin (GDF-8) and GDF-11, mediate Smad2/3 signaling through binding and activation of their high-affinity receptors ActRIIA and ActRIIB on the cell surface. The Activins and related proteins play a critical role in the regulation of a wide range of biology activities, including mesoderm induction, cell differentiation, myogenesis, bone remodeling, hematopoiesis, fibrogenesis, and reproductive physiology. Follistatin (FST), a secreted glycoprotein, binds to the Activins and Activin-related ligands to negatively control their signaling activities.

### Activin Ligands

In various embodiments, the bifunctional molecule of the present invention is capable of binding an Activin or Activin-related ligand having an amino acid sequence selected from the group consisting of the amino acid sequences set forth in SEQ ID NOs: 1-9:

Human ActRIIA-ECD
(SEQ ID NO: 1)
ETQECLFFNANWEKDRTNQTGVEPCYGDKDKRRHCFATWKNISGSIEIV
KQGCWLDDINCYDRTDCVEKKDSPEVYFCCCEGNMCNEKFSYFPEMEVT
QPTSNPVTPKPP
Human ActRIIB-ECD
(SEQ ID NO: 2)
ETRECIYYNANWELERTNQSGLERCEGEQDKRLHCYASWRNSSGTIELV
KKGCWLDDFNCYDRQECVATEENPQVYFCCCEGNFCNERFTHLPEAGGP
EV TYEPPPTAPT
Human Follistatin 315
(SEQ ID NO: 3)
GNCWLRQAKNGRCQVLYKTELSKEECCSTGRLSTSWTEEDVNDNTLFKW
MIFNGGAPNCIPCKETCENVDCGPGKKCRMNKKNKPRCVCAPDCSNITW
KGPVCGLDGKTYRNECALLKARCKEQPELEVQYQGRCKKTCRDVFCPGS
STCVVDQTNNAYCVTCNRICPEPASSEQYLCGNDGVTYSSACHLRKATC
LLGRSIGLAYEGKCIKAKSCEDIQCTGGKKCLWDFKVGRGRCSLCDELC
PDSKSDEPVCASDNATYASECAMKEAACSSGVLLEVKHSGSCNSISEDT
EEEEEDEDQDYSFPISSILEW
Human Follistatin AHBS (modified Follistatin)
(SEQ ID NO: 4)
GNCWLRQAKNGRCQVLYKTELSKEECCSTGRLSTSWTEEDVNDNTLFKW
MIFNGGAPNCIPCKETCENVDCGPGQSCVVDQTGSPRCVCAPDCSNITW
KGPVCGLDGKTYRNECALLKARCKEQPELEVQYQGRCKKTCRDVFCPGS
STCVVDQTNNAYCVTCNRICPEPASSEQYLCGNDGVTYSSACHLRKATC
LLGRSIGLAYEGKCIKAKSCEDIQCTGGKKCLWDFKVGRGRCSLCDELC
PDSKSDEPVCASDNATYASECAMKEAACSSGVLLEVKHSGSCNSISEDT
EEEEEDEDQDYSFPISSILEW
Human Follistatin 288
(SEQ ID NO: 5)
GNCWLRQAKNGRCQVLYKTELSKEECCSTGRLSTSWTEEDVNDNTLFKW
MIFNGGAPNCIPCKETCENVDCGPGKKCRMNKKNKPRCVCAPDCSNITW
KGPVCGLDGKTYRNECALLKARCKEQPELEVQYQGRCKKTCRDVFCPGS
STCVVDQTNNAYCVTCNRICPEPASSEQYLCGNDGVTYSSACHLRKATC
LLGRSIGLAYEGKCIKAKSCEDIQCTGGKKCLWDFKVGRGRCSLCDELC
PDSKSDEPVCASDNATYASECAMKEAACSSGVLLEVKHSGSCN
Modified human ActRIIB ECD
(SEQ ID NO: 6)
ETRECIYYNANWELERTNQSGLERCEGDQDKRLHCYASWRNSSGTIELV
KKGCWLDDINCYDRQECVATKENPQVYFCCCEGNFCNERFTHLPEAGGP
EVTYEPPPTAPT
Modified human ActRIIB ECD
(SEQ ID NO: 7)
ETRECIYYNANWELERTNQSGLERCYGDKDKRRHCYASWRNSSGTIELV
KKGCWLDDENCYDRQECVATEENPQVYFCCCEGNFCNERFTHLPEAGGP
EVTYEPPPTAPT
Modified human ActRIIB ECD
(SEQ ID NO: 8)
ETRECIYYNANWELERTNQSGLERCEGEQDKRLHCYASWRNSSGTIELV
KKGCWDDDFNCYDRQECVATEENPQVYFCCCEGNFCNERFTHLPEAGGP
EVTYEPPPT
Modified human ActRIIA ECD
(SEQ ID NO: 9)
GAILGRSETQECLFYNANWELERTNQTGVEPCEGEKDKRLHCYATWRNI
SGSIEIVKKGCWLDDFNCYDRTDCVETEENPQVYFCCCEGNMCNEKFSY
FPEMEVTQPTS

In various embodiments, the bifunctional polypeptide molecule is capable of binding an Activin or Activin-related ligand having an amino acid sequence selected from the group consisting of the amino acid sequences set forth in Table 2:

TABLE 2
Polypeptides containing Activin and Activin-related ligands

| LIGAND | DATABASE | ACCESSION NO |
|---|---|---|
| Activin A | UniProtKB | A4D1W7-1 |
| Activin B | UniProtKB | P09529-1 |
| GDF8 | UniProtKB | A1C2F0-1 |
| GDF11 | UniProtKB | O95390-1 |

### TGF-β Ligands

Transforming Growth Factor-Beta (TGF-β), including TGF-β1, TGF-β2 and TGF-β3, mediates Smad2/3 signaling through its binding and activation of the high-affinity receptors TGFβRII and TGFβRIIB on the cell surface. TGF-β plays a critical role in the regulation of a wide range of biology activities, including immune function, cell proliferation and differentiation, epithelial-mesenchymal transition, fibrogenesis, hematopoiesis, myogenesis, bone remodeling, cancer progression and metastasis. Elevated TGF-β levels and consequently increased Smad2/3 signaling have been implicated in pathogenesis and progression of many disease conditions including cancer, anemia, bone metastasis, bone loss, fibrosis, pain, muscle loss, insulin resistance, chronic kidney disease, liver disease, and cardiovascular diseases.

In various embodiments, the bifunctional polypeptide molecule of the present invention is capable of binding an TGF-β ligand having an amino acid sequence selected from the group consisting of the amino acid sequences set forth in SEQ ID NOs: 18-19:

Human TGF-β Receptor II-ECD isoform 1 (TGF-β
RIIB-ECD)
(SEQ ID NO: 18)
TIPPHVQKSVNNDMIVTDNNGAVKFPQLCKFCDVRFSTCDNQKSCMSNC
SITSICEKPQEVCVAVWRKNDENITLETVCHDPKLPYHDFILEDAASPK
CIMKEKKKPGETFFMCSCSSDECNDNIIFSEEYNTSNPD
Human TGF-β Receptor II-ECD isoform 2 (TGF-β
RIIA-ECD)
(SEQ ID NO: 19)
TIPPHVQKSDVEMEAQKDEIICPSCNRTAHPLRHINNDMIVTDNNGAVK
FPQLCKFCDVRFSTCDNQKSCMSNCSITSICEKPQEVCVAVWRKNDENI
TLETVCHDPKLPYHDFILEDAASPKCIMKEKKKPGETFFMCSCSSDECN
DNIIFSEEYNTSNPD

In various embodiments, the bifunctional polypeptide molecule is capable of binding a TGF-β ligand having an amino acid sequence selected from the group consisting of the amino acid sequences set forth in Table 3:

TABLE 3
Polypeptides containing TGF-β ligands

| LIGAND | DATABASE | ACCESSION NO |
|---|---|---|
| TGF-β1 | UniProtKB | P01137-1 |
| TGF-β2 | UniProtKB | P61812-1 |
| TGF-β3 | UniProtKB | P10600-1 |

### RANKL

The receptor activator of nuclear factor-kappa B ligand (RANKL) signaling pathway plays a central role in the regulation of bone mass by stimulating osteoclast activity and increasing bone resorption, even though RANKL has no direct influence on osteoblast activity and bone formation. The RANKL signaling pathway can also powerfully regulate bone mass. Mounting evidence indicates that the RANKL signaling pathway is increased in various skeletal disorders, indicating their parallel contribution to pathogenesis of bone disorders. Importantly, activation of the RANKL signaling pathway can strongly induce epithelial-mesenchymal transition (EMT), a remodeling process that is critical for metastasis. In fact, expression levels of RANKL has been shown to be elevated in bone metastasis, suggesting that increased RANKL and Activin/TGF-β signaling activities act in parallel to drive pathogenesis and progression of bone metastatic diseases in cancer. In addition, muscle mass plays an important role in maintaining healthy bone. It has been shown that in wasting disease states, such as cancer cachexia, age-related sarcopenia and neuromuscular diseases, muscle wasting is intimately correlated with bone loss. Recent studies suggest that increased RANKL signaling can trigger muscle loss.

In various embodiments, the bifunctional antagonist molecule is capable of binding a RANKL having an amino acid sequence selected from the group consisting of the amino acid sequences set forth in Table 4:

TABLE 4
Polypeptides containing RANKL

| LIGAND | DATABASE | ACCESSION NO |
|---|---|---|
| RANKL | UniProtKB | Q9Y6Q6-1 |
| RANKL | UniProtKB | Q9Y6Q6-2 |
| RANKL | UniProtKB | Q9Y6Q6-3 |
| RANKL | UniProtKB | Q9Y6Q6-4 |
| RANKL | UniProtKB | Q9Y6Q6-5 |
| RANKL | UniProtKB | Q9Y6Q6-6 |
| OPGL | UniProtKB | O14788-1 |

### RANKL and/or Activin and/or TGF-β Antibodies and Antibody Fragments

Methods of generating novel antibodies that bind to RANKL and/or Activin or Activin-related ligand and/or TGF-β ligands and/or receptors are known to those skilled in the art. For example, a method for generating a monoclonal antibody that binds specifically to RANKL and/or Activin or Activin-related ligand and/or TGF-β ligand may comprise administering to a mouse an amount of an immunogenic composition comprising RANKL and/or Activin or Activin-related ligand and/or TGF-β ligand effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to RANKL and/or Activin or Activin-related ligand and/or TGF-β ligand. Once obtained, a hybridoma can be propagated in a cell culture, optionally in culture conditions where the hybridoma-derived cells produce the monoclonal antibody that binds specifically to RANKL and/or Activin or Activin-related ligand and/or TGF-β ligand. The monoclonal antibody may be purified from the cell culture. A variety of different techniques are then available for testing an antigen/antibody interaction to identify particularly desirable antibodies.

Other suitable methods of producing or isolating antibodies of the requisite specificity can used, including, for example, methods which select recombinant antibody from a library, or which rely upon immunization of transgenic animals (e.g., mice) capable of producing a full repertoire of human antibodies. See e.g., Jakobovits et al., Proc. Natl. Acad. Sci. (U.S.A.), 90: 2551-2555, 1993; Jakobovits et al., Nature, 362: 255-258, 1993; Lonberg et al., U.S. Pat. No. 5,545,806; and Surani et al., U.S. Pat. No. 5,545,807.

Antibodies can be engineered in numerous ways. They can be made as single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science, 240:1041-1043, 1988; Liu et al., Proc. Natl. Acad. Sci. (U.S.A.), 84:3439-3443, 1987; Liu et al., J. Immunol., 139:3521-3526, 1987; Sun et al., Proc. Natl. Acad. Sci. (U.S.A.), 84:214-218, 1987; Nishimura et al., Canc. Res., 47:999-1005, 1987; Wood et al., Nature, 314:446-449, 1985; and Shaw et al., J. Natl Cancer Inst., 80:1553-1559, 1988).

Methods for humanizing antibodies have been described in the art. In some embodiments, a humanized antibody has one or more amino acid residues introduced from a source that is nonhuman, in addition to the nonhuman CDRs. Humanization can be essentially performed following the method of Winter and co-workers (Jones et al., Nature, 321:522-525, 1986; Riechmann et al., Nature, 332:323-327, 1988; Verhoeyen et al., Science, 239:1534-1536, 1988), by substituting hypervariable region sequences for the corresponding sequences of a human antibody. Accordingly, such “humanized” antibodies are chimeric antibodies (U.S. Pat. No. 4,816,567) wherein substantially less than an intact human variable region has been substituted by the corresponding sequence from a nonhuman species. In practice, humanized antibodies are typically human antibodies in which some hypervariable region residues and possibly some framework region residues are substituted by residues from analogous sites in rodent antibodies.

U.S. Pat. No. 5,693,761 to Queen et al, discloses a refinement on Winter et al. for humanizing antibodies, and is based on the premise that ascribes avidity loss to problems in the structural motifs in the humanized framework which, because of steric or other chemical incompatibility, interfere with the folding of the CDRs into the binding-capable conformation found in the mouse antibody. To address this problem, Queen teaches using human framework sequences closely homologous in linear peptide sequence to framework sequences of the mouse antibody to be humanized. Accordingly, the methods of Queen focus on comparing framework sequences between species. Typically, all available human variable region sequences are compared to a particular mouse sequence and the percentage identity between correspondent framework residues is calculated. The human variable region with the highest percentage is selected to provide the framework sequences for the humanizing project. Queen also teaches that it is important to retain in the humanized framework, certain amino acid residues from the mouse framework critical for supporting the CDRs in a binding-capable conformation. Potential criticality is assessed from molecular models. Candidate residues for retention are typically those adjacent in linear sequence to a CDR or physically within 6 Å of any CDR residue.

Another method of humanizing antibodies, referred to as “framework shuffling”, relies on generating a combinatorial library with nonhuman CDR variable regions fused in frame into a pool of individual human germline frameworks (Dall'Acqua et al., Methods, 36:43, 2005). The libraries are then screened to identify clones that encode humanized antibodies which retain good binding.

Methods for making fully human antibodies have been described in the art. By way of example, a method for producing an anti-Activin antibody or antigen-binding fragment thereof comprises the steps of synthesizing a library of human antibodies on phage, screening the library with Activin polypeptide or an antibody-binding portion thereof, isolating phage that bind Activin polypeptide, and obtaining the antibody from the phage. By way of another example, one method for preparing the library of antibodies for use in phage display techniques comprises the steps of immunizing a non-human animal comprising human immunoglobulin loci with Activin polypeptide or an antigenic portion thereof to create an immune response, extracting antibody-producing cells from the immunized animal; isolating RNA encoding heavy and light chains of antibodies of the invention from the extracted cells, reverse transcribing the RNA to produce cDNA, amplifying the cDNA using primers, and inserting the cDNA into a phage display vector such that antibodies are expressed on the phage. Recombinant anti- Activin antibodies of the invention may be obtained in this way.

Recombinant human anti-RANKL and/or anti-Activin and/or anti-TGF-β antibodies of the invention can also be isolated by screening a recombinant combinatorial antibody library. Preferably the library is a scFv phage display library, generated using human V_{L} and V_{H} cDNAs prepared from mRNA isolated from B cells. Methods for preparing and screening such libraries are known in the art. Kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology, 9:1370-1372 (1991); Hay et al., Hum. Antibod. Hybridomas, 3:81-85, 1992; Huse et al., Science, 246:1275-1281, 1989; McCafferty et al., Nature, 348:552-554, 1990; Griffiths et al., EMBO J., 12:725-734, 1993; Hawkins et al., J. Mol. Biol., 226:889-896, 1992; Clackson et al., Nature, 352:624-628, 1991; Gram et al., Proc. Natl. Acad. Sci. (U.S.A.), 89:3576-3580, 1992; Garrad et al., Bio/Technology, 9:1373-1377, 1991; Hoogenboom et al., Nuc. Acid Res., 19:4133-4137, 1991; and Barbas et al., Proc. Natl. Acad. Sci. (U.S.A.), 88:7978-7982, 1991), all incorporated herein by reference.

Human antibodies are also produced by immunizing a non-human, transgenic animal comprising within its genome some or all of human immunoglobulin heavy chain and light chain loci with a human IgE antigen, e.g., a XenoMouse™ animal (Abgenix, Inc./Amgen, Inc.—Fremont, Calif.). XenoMouse™ mice are engineered mouse strains that comprise large fragments of human immunoglobulin heavy chain and light chain loci and are deficient in mouse antibody production. See, e.g., Green et al., Nature Genetics, 7:13-21, 1994 and U.S. Pat. Nos. 5,916,771, 5,939,598, 5,985,615, 5,998,209, 6,075,181, 6,091,001, 6,114,598, 6,130,364, 6,162,963 and 6,150,584. XenoMouse™ mice produce an adult-like human repertoire of fully human antibodies and generate antigen-specific human antibodies. In some embodiments, the XenoMouse™ mice contain approximately 80% of the human antibody V gene repertoire through introduction of megabase sized, germline configuration fragments of the human heavy chain loci and kappa light chain loci in yeast artificial chromosome (YAC). In other embodiments, XenoMouse™ mice further contain approximately all of the human lambda light chain locus. See Mendez et al., Nature Genetics, 15:146-156, 1997; Green and Jakobovits, J. Exp. Med., 188:483-495, 1998; and WO 98/24893. In one aspect, the present invention provides a method for making anti-RANKL and/or anti-Activin and/or TGF-β antibodies from non-human, non-mouse animals by immunizing non-human transgenic animals that comprise human immunoglobulin loci with RANKL and/or Activin and/or TGF-β polypeptide. One can produce such animals using the methods described in the above-cited documents.

### Anti-Activin Antibodies

In various embodiments of the present invention, the anti-Activin antibody is an anti-Activin A antibody that is a human antibody or antigen-binding fragment comprising the heavy chain amino acid sequence set forth in SEQ ID NO: 10:

(SEQ ID NO: 10)
QVQLVQSGAEVKKPGASVKVSCKASGYTFTSYGLSWVRQAPGQGLEWMG
WIIPYNGNTNSAQKLQGRVTMTTDTSTSTAYMELRSLRSDDTAVYFCAR
DRDYGVNYDAFDIWGQGTMVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNVYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHY
TQKSLSLSPGK

a human antibody or antigen-binding fragment comprising the light chain amino acid sequence set forth in SEQ ID NO: 11:

(SEQ ID NO: 11)
SYEVTQAPSVSVSPGQTASITCSGDKLGDKYACWYQQKPGQSPVLVIYQ
DSKRPSGIPERFSGSNSGNTATLTISGTQAMDEADYYCQAWDSSTAVFG
GGTKLTVLRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

or a human antibody or antigen-binding fragment comprising the heavy chain amino acid sequence set forth in SEQ ID NO: 10 and the light chain amino acid sequence set forth in SEQ ID NO: 11;
a human antibody or antigen-binding fragment comprising the heavy chain variable region amino acid sequence set forth in SEQ ID NO: 12:

(SEQ ID NO: 12)
QVQLVQSGAEVKKPGASVKVSCKASGYTFTSYGLSWVRQAPGQGLEWMG
WIIPYNGNTNSAQKLQGRVTMTTDTSTSTAYMELRSLRSDDTAVYFCAR
DRDYGVNYDAFDIWGQGTMVTVSS

a human antibody or antigen-binding fragment comprising the light chain variable region amino acid sequence set forth in SEQ ID NO: 13:

(SEQ ID NO: 13)
SYEVTQAPSVSVSPGQTASITCSGDKLGDKYACWYQQKPGQSPVLVIYQ
DSKRPSGIPERFSGSNSGNTATLTISGTQAMDEADYYCQAWDSSTAVFG
GGTKLTVL

or a human antibody or antigen-binding fragment comprising the heavy chain variable region amino acid sequence set forth in SEQ ID NO: 12 and the light chain variable region amino acid sequence set forth in SEQ ID NO: 13.

In various embodiments, the invention provides antibodies, comprising a heavy chain, a light chain, or both a heavy chain and light chain; a heavy chain variable region, a light chain variable region, or both a heavy chain variable region and light chain variable region; wherein the heavy chain, light chain, heavy chain variable region, or light chain variable region comprises a sequence that has at least about 75%, at least about 80%, at least about 85%, at least about 90%, 91%, 92%, 93%, 94%, 95%, 96%, 97%, 98%, or at least about 99% identity to the amino acid sequences as set forth in SEQ ID NOs: 10, 11, 12 or 13; wherein the antibody binds specifically to human Activin A.

In various embodiments of the present invention, the anti-Activin antibody is an anti-Activin A antibody that is a human antibody or antigen-binding fragment comprising the heavy chain amino acid sequence set forth in SEQ ID NO: 14:

(SEQ ID NO: 14)
QVQLQESGPGLVKPSETLSLTCTVSGGSFSSHFWSWIRQPPGKGLEWIG
YILYTGGTSFNPSLKSRVSMSVGTSKNQFSLKLSSVTAADTAVYYCARA
RSGITFTGIIVPGSFDIWGQGTMVTVSSASTKGPSVFPLAPCSRSTSES
TAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNH
YTQKSLSLSLGK

a human antibody or antigen-binding fragment comprising the light chain amino acid sequence set forth in SEQ ID NO: 15:

(SEQ ID NO: 15)
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

or a human antibody or antigen-binding fragment comprising the heavy chain amino acid sequence set forth in SEQ ID NO: 14 and the light chain amino acid sequence set forth in SEQ ID NO: 15;
a human antibody or antigen-binding fragment comprising the heavy chain variable region amino acid sequence set forth in SEQ ID NO: 16:

(SEQ ID NO: 16)
QVQLQESGPGLVKPSETLSLTCTVSGGSFSSHFWSWIRQPPGKGLEWIG
YILYTGGTSFNPSLKSRVSMSVGTSKNQFSLKLSSVTAADTAVYYCARA
RSGITFTGIIVPGSFDIWGQGTMVTVSS

a human antibody or antigen-binding fragment comprising the light chain variable region amino acid sequence set forth in SEQ ID NO: 17:

(SEQ ID NO: 17)
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPWT
FGQGTKVEIK

or a human antibody or antigen-binding fragment comprising the heavy chain variable region amino acid sequence set forth in SEQ ID NO: 16 and the light chain variable region amino acid sequence set forth in SEQ ID NO: 17.

In various embodiments, the invention provides antibodies, comprising a heavy chain, a light chain, or both a heavy chain and light chain; a heavy chain variable region, a light chain variable region, or both a heavy chain variable region and light chain variable region; wherein the heavy chain, light chain, heavy chain variable region, or light chain variable region comprises a sequence that has at least about 75%, at least about 80%, at least about 85%, at least about 90%, 91%, 92%, 93%, 94%, 95%, 96%, 97%, 98%, or at least about 99% identity to the amino acid sequences as set forth in SEQ ID NOs: 14, 15, 16 or 17; wherein the antibody binds specifically to human Activin A.

### Anti-TGF-β Antibodies

In various embodiments of the present invention, the anti-TGF-β antibody is an anti-TGF-β antibody that is a human antibody or antigen-binding fragment comprising the heavy chain amino acid sequence set forth in SEQ ID NO: 20:

(SEQ ID NO: 20)
QVQLVQSGAEVKKPGSSVKVSCKASGYTFSSNVISWVRQAPGQGLEWMG
GVIPIVDIANYAQRFKGRVTITADESTSTTYMELSSLRSEDTAVYYCAS
TLGLVLDAMDYWGQGTLVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPSCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLGK

a human antibody or antigen-binding fragment comprising the light chain amino acid sequence set forth in SEQ ID NO: 21:

(SEQ ID NO: 21)
ETVLTQSPGTLSLSPGERATLSCRASQSLGSSYLAWYQQKPGQAPRLLI
YGASSRAPGIPDRFSGSGSGTDFTLTISRLEPEDFAVYYCQQYADSPIT
FGQGTRLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

or a human antibody or antigen-binding fragment comprising the heavy chain amino acid sequence set forth in SEQ ID NO: 20 and the light chain amino acid sequence set forth in SEQ ID NO: 21;
a human antibody or antigen-binding fragment comprising the heavy chain variable region amino acid sequence set forth in SEQ ID NO: 22:

(SEQ ID NO: 22)
QVQLVQSGAEVKKPGSSVKVSCKASGYTFSSNVISWVRQAPGQGLEWMG
GVIPIVDIANYAQRFKGRVTITADESTSTTYMELSSLRSEDTAVYYCAS
TLGLVLDAMDYWGQGTLVTVSS

a human antibody or antigen-binding fragment comprising the light chain variable region amino acid sequence set forth in SEQ ID NO: 23:

(SEQ ID NO: 23)
ETVLTQSPGTLSLSPGERATLSCRASQSLGSSYLAWYQQKPGQAPRLLI
YGASSRAPGIPDRFSGSGSGTDFTLTISRLEPEDFAVYYCQQYADSPIT
FGQGTRLEIK

or a human antibody or antigen-binding fragment comprising the heavy chain variable region amino acid sequence set forth in SEQ ID NO: 22 and the light chain variable region amino acid sequence set forth in SEQ ID NO: 23.

In various embodiments, the invention provides antibodies, comprising a heavy chain, a light chain, or both a heavy chain and light chain; a heavy chain variable region, a light chain variable region, or both a heavy chain variable region and light chain variable region; wherein the heavy chain, light chain, heavy chain variable region, or light chain variable region comprises a sequence that has at least about 75%, at least about 80%, at least about 85%, at least about 90%, 91%, 92%, 93%, 94%, 95%, 96%, 97%, 98%, or at least about 99% identity to the amino acid sequences as set forth in SEQ ID NOs: 20, 21, 22 or 23; wherein the antibody binds specifically to human TGF-β.

### RANKL Antibodies

In various embodiments of the present invention, the anti-RANKL antibody is an anti-RANKL antibody that is a human antibody or antigen-binding fragment comprising the heavy chain amino acid sequence set forth in SEQ ID NO: 24:

(SEQ ID NO: 24)
EVQLLESGGGLVQPGGSLRLSCAASGFTFSSYAMSWVRQAPGKGLEWVS
GITGSGGSTYYADSVKGRFTISRDNSKNTLYLQMNSLRAEDTAVYYCAK
DPGTTVIMSWFDPWGQGTLVTVSSASTKGPSVFPLAPCSRSTSESTAAL
GCLVKDYFPEPVTVSWNSGALTSGVHTFPAVLQSSGLYSLSSVVTVPSS
NFGTQTYTCNVDHKPSNTKVDKTVERKCCVECPPCPAPPVAPSVFLFPP
KPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYK
TTPPMLDSDGSFFLYSKLTVDKSRWQQGNVFSCSVMHEALHNHYTQKSL
SLSPGK

a human antibody or antigen-binding fragment comprising the light chain amino acid sequence set forth in SEQ ID NO: 25:

(SEQ ID NO: 25)
EIVLTQSPGTLSLSPGERATLSCRASQSVRGRYLAWYQQKPGQAPRLLI
YGASSRATGIPDRFSGSGSGTDFTLTISRLEPEDFAVFYCQQYGSSPRT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

or a human antibody or antigen-binding fragment comprising the heavy chain amino acid sequence set forth in SEQ ID NO: 24 and the light chain amino acid sequence set forth in SEQ ID NO: 25;
a human antibody or antigen-binding fragment comprising the heavy chain variable region amino acid sequence set forth in SEQ ID NO: 26:

(SEQ ID NO: 26)
EVQLLESGGGLVQPGGSLRLSCAASGFTFSSYAMSWVRQAPGKGLEWVS
GITGSGGSTYYADSVKGRFTISRDNSKNTLYLQMNSLRAEDTAVYYCAK
DPGTTVIMSWFDPWGQGTLVTVSS

a human antibody or antigen-binding fragment comprising the light chain variable region amino acid sequence set forth in SEQ ID NO: 27:

(SEQ ID NO: 27)
EIVLTQSPGTLSLSPGERATLSCRASQSVRGRYLAWYQQKPGQAPRLLI
YGASSRATGIPDRFSGSGSGTDFTLTISRLEPEDFAVFYCQQYGSSPRT
FGQGTKVEIK

or a human antibody or antigen-binding fragment comprising the heavy chain variable region amino acid sequence set forth in SEQ ID NO: 26 and the light chain variable region amino acid sequence set forth in SEQ ID NO: 27.

In various embodiments, the invention provides antibodies, comprising a heavy chain, a light chain, or both a heavy chain and light chain; a heavy chain variable region, a light chain variable region, or both a heavy chain variable region and light chain variable region; wherein the heavy chain, light chain, heavy chain variable region, or light chain variable region comprises a sequence that has at least about 75%, at least about 80%, at least about 85%, at least about 90%, 91%, 92%, 93%, 94%, 95%, 96%, 97%, 98%, or at least about 99% identity to the amino acid sequences as set forth in SEQ ID NOs: 24, 25, 26 or 27; wherein the antibody binds specifically to human RANKL.

In various embodiments, the RANKL-Binding Polypeptide is human osteoprotegerin (OPG) comprising the amino acid sequence set forth in SEQ ID NO: 28.

(SEQ ID NO: 28)
MNNLLCCALVFLDISIKWTTQETFPPKYLHYDEETSHQLLCDKCPPGTY
LKQHCTAKWKTVCAPCPDHYYTDSWHTSDECLYCSPVCKELQYVKQECN
RTHNRVCECKEGRYLEIEFCLKHRSCPPGFGVVQAGTPERNTVCKRCPD
GFFSNETSSKAPCRKHTNCSVFGLLLTQKGNATHDNICSGNSESTQKCG
IDVTLCEEAFFRFAVPTKFTPNWLSVLVDNLPGTKVNAESVERIKRQHS
SQEQTFQLLKLWKHQNKDQDIVKKIIQDIDLCENSVQRHIGHANLTFEQ
LRSLMESLPGKKVGAEDIEKTIKACKPSDQILKLLSLWRIKNGDQDTLK
GLMHALKHSKTYHFPKTVTQSLKKTIRFLHSFTMYKLYQKLFLEMIGNQ
VQSVKISCL

### Bifunctional Antagonist Molecules

In one aspect, the present invention provides novel polypeptide-based bifunctional antagonist molecules specifically designed to simultaneously neutralize RANKL signaling and either Activin signaling and TGF-β signaling in a potent manner and comprising a first antigen-binding molecule that specifically binds to RANKL and a second antigen-binding molecule that specifically binds to either Activin ligand or TGF-β ligand. In various embodiments, the bifunctional antagonist molecule comprises an isolated antibody, or antigen-binding fragment thereof, that specifically binds to RANKL and an isolated antibody, or antigen-binding fragment thereof, that specifically binds to either Activin ligand or TGF-β ligand. Importantly, these bifunctional antagonists also provide advantageous properties such as producibility, stability, binding affinity, biological activity, specific targeting of certain cells, targeting efficiency and reduced toxicity.

### Exemplary Bifunctional Antagonist Molecules

In various embodiments, the bifunctional antagonist molecules of the present invention are selected from the group of molecules designed and comprising the fusion partners as described in Table 5:

TABLE 5

| Fusion Partner A (RANK or | | Fusion Partner B (Activin or |
|---|---|---|
| OPG-Binding Polypeptide) | Linker | TGF-β-Binding Polypeptide) |
| SEQ ID NO: | SEQ ID NOs: | SEQ ID NO: |
| SEQ ID NO: 26 and SEQ ID NO: 27 | 60-79 | SEQ ID NO: 1 |
| SEQ ID NO: 26 and SEQ ID NO: 27 | 60-79 | SEQ ID NO: 2 |
| SEQ ID NO: 26 and SEQ ID NO: 27 | 60-79 | SEQ ID NO: 3 |
| SEQ ID NO: 26 and SEQ ID NO: 27 | 60-79 | SEQ ID NO: 4 |
| SEQ ID NO: 26 and SEQ ID NO: 27 | 60-79 | SEQ ID NO: 5 |
| SEQ ID NO: 26 and SEQ ID NO: 27 | 60-79 | SEQ ID NO: 6 |
| SEQ ID NO: 26 and SEQ ID NO: 27 | 60-79 | SEQ ID NO: 7 |
| SEQ ID NO: 26 and SEQ ID NO: 27 | 60-79 | SEQ ID NO: 8 |
| SEQ ID NO: 26 and SEQ ID NO: 27 | 60-79 | SEQ ID NO: 9 |
| SEQ ID NO: 26 and SEQ ID NO: 27 | 60-79 | SEQ ID NO: 12 and SEQ ID NO: 13 |
| SEQ ID NO: 26 and SEQ ID NO: 27 | 60-79 | SEQ ID NO: 16 and SEQ ID NO: 17 |
| SEQ ID NO: 26 and SEQ ID NO: 27 | 60-79 | SEQ ID NO: 18 |
| SEQ ID NO: 26 and SEQ ID NO: 27 | 60-79 | SEQ ID NO: 19 |
| SEQ ID NO: 26 and SEQ ID NO: 27 | 60-79 | SEQ ID NO: 22 and SEQ ID NO: 23 |
| SEQ ID NO: 28 | 60-79 | SEQ ID NO: 1 |
| SEQ ID NO: 28 | 60-79 | SEQ ID NO: 2 |
| SEQ ID NO: 28 | 60-79 | SEQ ID NO: 3 |
| SEQ ID NO: 28 | 60-79 | SEQ ID NO: 4 |
| SEQ ID NO: 28 | 60-79 | SEQ ID NO: 5 |
| SEQ ID NO: 28 | 60-79 | SEQ ID NO: 6 |
| SEQ ID NO: 28 | 60-79 | SEQ ID NO: 7 |
| SEQ ID NO: 28 | 60-79 | SEQ ID NO: 8 |
| SEQ ID NO: 28 | 60-79 | SEQ ID NO: 9 |
| SEQ ID NO: 12 and SEQ ID NO: 13 | 60-79 | SEQ ID NO: 28 |
| SEQ ID NO: 16 and SEQ ID NO: 17 | 60-79 | SEQ ID NO: 28 |
| SEQ ID NO: 28 | 60-79 | SEQ ID NO: 18 |
| SEQ ID NO: 28 | 60-79 | SEQ ID NO: 19 |
| SEQ ID NO: 22 and SEQ ID NO: 23 | 60-79 | SEQ ID NO: 28 |

In various embodiments, the bifunctional antagonist molecule of the present invention specifically designed to simultaneously neutralize Activin signaling and OPG signaling in a potent manner and comprising a first antigen-binding molecule that specifically binds to Activin ligand and a second antigen-binding molecule that specifically binds to OPG ligand is selected from the group of molecules as described in Table 6:

TABLE 6

| Bifunctional Antagonist Molecule | SEQ ID NO: |
|---|---|
| A242 | SEQ ID NO: 40 |
| A243 | SEQ ID NO: 41 |
| A244 | SEQ ID NO: 42 |
| A245 | SEQ ID NO: 43 |
| A246 | SEQ ID NO: 44 |
| A247 | SEQ ID NO: 45 |
| A248 | SEQ ID NO: 46 |
| A249 | SEQ ID NO: 47 |
| A250 | SEQ ID NO: 48 |

In various embodiments, the bifunctional antagonist molecule of the present invention specifically designed to simultaneously neutralize Activin signaling and OPG signaling in a potent manner and comprising a first antigen-binding molecule that specifically binds to TGF-β ligand and a second antigen-binding molecule that specifically binds to OPG ligand is selected from the group of molecules as described in Table 7:

TABLE 7

| Bifunctional Antagonist Molecule | SEQ ID NO: |
|---|---|
| A253 | SEQ ID NO: 51 |
| A254 | SEQ ID NO: 52 |

### Linkers

In various embodiments, the first antigen-binding molecule that specifically binds to RANKL is attached to the second antigen-binding molecule that specifically binds to either Activin ligand or TGF-β ligand by a linker and/or a hinge linker peptide. The linker or hinge linker may be an artificial sequence of between 5, 10, 15, 20, 30, 40 or more amino acids that are relatively free of secondary structure or display α-helical conformation.

Peptide linker provides covalent linkage and additional structural and/or spatial flexibility between protein domains. As known in the art, peptide linkers contain flexible amino acid residues, such as glycine and serine. In various embodiments, peptide linker may include 1-100 amino acids. In various embodiments, a spacer can contain motif of GGGSGGGS (SEQ ID NO: 67). In other embodiments, a linker can contain motif of GGGGS (SEQ ID NO: 70)n, wherein n is an integer from 1 to 10. In other embodiments, a linker can also contain amino acids other than glycine and serine. In another embodiment, a linker can contain other protein motifs, including but not limited to, sequences of α-helical conformation such as AEAAAKEAAAKEAAAKA (SEQ ID NO: 65). In various embodiments, linker length and composition can be tuned to optimize activity or developability, including but not limited to, expression level and aggregation propensity. In another embodiment, the peptide linker can be a simple chemical bond, e.g., an amide bond (e.g., by chemical conjugation of PEG).

Exemplary peptide linkers are provided in Table 8:

TABLE 8

| Linker Sequence | SEQ ID NO: |
|---|---|
| GGGSGGGSGGGS | 60 |
| GGGS | 61 |
| GSSGGSGGSGGSG | 62 |
| GSSGT | 63 |
| GGGGSGGGGSGGGS | 64 |
| AEAAAKEAAAKEAAAKA | 65 |
| GGGGSGGGGSGGGGSGGGGS | 66 |
| GGGSGGGS | 67 |
| GSGST | 68 |
| GGSS | 69 |
| GGGGS | 70 |
| GGSG | 71 |
| SGGG | 72 |
| GSGS | 73 |
| GSGSGS | 74 |
| GSGSGSGS | 75 |
| GSGSGSGSGS | 76 |
| GSGSGSGSGSGS | 77 |
| GGGGSGGGGS | 78 |
| GGGGSGGGGGGGGS | 79 |

### Polynucleotides

In another aspect, the present disclosure provides isolated nucleic acid molecules comprising a polynucleotide encoding a bifunctional antagonist molecule of the present disclosure. The subject nucleic acids may be single-stranded or double stranded. Such nucleic acids may be DNA or RNA molecules. DNA includes, for example, cDNA, genomic DNA, synthetic DNA, DNA amplified by PCR, and combinations thereof. Genomic DNA encoding bifunctional antagonist molecules is obtained from genomic libraries which are available for a number of species. Synthetic DNA is available from chemical synthesis of overlapping oligonucleotide fragments followed by assembly of the fragments to reconstitute part or all of the coding regions and flanking sequences. RNA may be obtained from prokaryotic expression vectors which direct high-level synthesis of mRNA, such as vectors using T7 promoters and RNA polymerase. cDNA is obtained from libraries prepared from mRNA isolated from various tissues that express a bifunctional antagonist molecule. The DNA molecules of the disclosure include full-length genes as well as polynucleotides and fragments thereof. The full-length gene may also include sequences encoding the N-terminal signal sequence.

In various embodiments, the isolated nucleic acid molecules comprise the polynucleotides described herein, and further comprise a polynucleotide encoding at least one heterologous protein described herein. In various embodiments, the nucleic acid molecules further comprise polynucleotides encoding the linkers or hinge linkers described herein.

In various embodiments, the recombinant nucleic acids of the present disclosure may be operably linked to one or more regulatory nucleotide sequences in an expression bifunctional antagonist molecule. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the present disclosure. The promoters may be either naturally occurring promoters, or hybrid promoters that combine elements of more than one promoter. An expression construct may be present in a cell on an episome, such as a plasmid, or the expression construct may be inserted in a chromosome. In various embodiments, the expression vector contains a selectable marker gene to allow the selection of transformed host cells. Selectable marker genes are well known in the art and will vary with the host cell used.

In another aspect of the present disclosure, the subject nucleic acid is provided in an expression vector comprising a nucleotide sequence encoding a bifunctional antagonist molecule and operably linked to at least one regulatory sequence. The term “expression vector” refers to a plasmid, phage, virus or vector for expressing a polypeptide from a polynucleotide sequence. Vectors suitable for expression in host cells are readily available and the nucleic acid molecules are inserted into the vectors using standard recombinant DNA techniques. Such vectors can include a wide variety of expression control sequences that control the expression of a DNA sequence when operatively linked to it may be used in these vectors to express DNA sequences encoding a bifunctional antagonist molecule. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda , the control regions for fd coat protein, the promoter for 3-phosphoglycerate kinase or other glycolytic enzymes, the promoters of acid phosphatase, e.g., PhoS, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant bifunctional antagonist molecule include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

In various embodiments, a vector will be designed for production of the subject bifunctional antagonist molecule in CHO cells, such as a Pcmv-Script vector (Stratagene, La Jolla, Calif.), pcDNA4 vectors (Invitrogen, Carlsbad, Calif.) and pCl-neo vectors (Promega, Madison, Wis.). As will be apparent, the subject gene constructs can be used to cause expression of the subject bifunctional antagonist molecule in cells propagated in culture, e.g., to produce proteins, including fusion proteins or variant proteins, for purification.

Accordingly, the present disclosure further pertains to methods of producing the subject bifunctional antagonist molecules. For example, a host cell transfected with an expression vector encoding a bifunctional antagonist molecule can be cultured under appropriate conditions to allow expression of the bifunctional antagonist molecule to occur. The bifunctional antagonist molecule may be secreted and isolated from a mixture of cells and medium containing the bifunctional antagonist molecule. Alternatively, the bifunctional antagonist molecule may be retained cytoplasmically or in a membrane fraction and the cells harvested, lysed and the protein isolated. A cell culture includes host cells, media and other byproducts. Suitable media for cell culture are well known in the art.

The polypeptides and proteins of the present disclosure can be purified according to protein purification techniques are well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The term “isolated polypeptide” or “purified polypeptide” as used herein, is intended to refer to a composition, isolatable from other components, wherein the polypeptide is purified to any degree relative to its naturally obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography; isoelectric focusing; gel electrophoresis; and combinations of these techniques. As is generally known in the art, it is believed that the order of conducting the various purification steps may be changed, or that certain steps may be omitted, and still result in a suitable method for the preparation of a substantially purified polypeptide.

### Pharmaceutical Compositions

In another aspect, the present disclosure provides a pharmaceutical composition comprising the isolated bifunctional antagonist molecules in admixture with a pharmaceutically acceptable carrier. Such pharmaceutically acceptable carriers are well known and understood by those of ordinary skill and have been extensively described (see, e.g., Remington's Pharmaceutical Sciences, 18th Edition, A. R. Gennaro, ed., Mack Publishing Company, 1990). The pharmaceutically acceptable carriers may be included for purposes of modifying, maintaining or preserving, for example, the pH, osmolarity, viscosity, clarity, color, isotonicity, odor, sterility, stability, rate of dissolution or release, adsorption or penetration of the composition. Such pharmaceutical compositions may influence the physical state, stability, rate of in vivo release, and rate of in vivo clearance of the polypeptide. Suitable pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

When parenteral administration is contemplated, the therapeutic pharmaceutical compositions may be in the form of a pyrogen-free, parenterally acceptable aqueous solution comprising the desired bifunctional antagonist molecule in a pharmaceutically acceptable vehicle. A particularly suitable vehicle for parenteral injection is sterile distilled water in which a polypeptide is formulated as a sterile, isotonic solution, properly preserved. In various embodiments, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Optionally, the suspension may also contain suitable stabilizers or agents to increase the solubility of the compounds and allow for the preparation of highly concentrated solutions.

In various embodiments, the therapeutic pharmaceutical compositions may be formulated for targeted delivery using a colloidal dispersion system. Colloidal dispersion systems include macromolecule complexes, nanocapsules, microspheres, beads, and lipid-based systems including oil-in-water emulsions, micelles, mixed micelles, and liposomes. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

In various embodiments, oral administration of the pharmaceutical compositions is contemplated. Pharmaceutical compositions that are administered in this fashion can be formulated with or without those carriers customarily used in the compounding of solid dosage forms such as tablets and capsules. In solid dosage forms for oral administration (capsules, tablets, pills, dragees, powders, granules, and the like), one or more therapeutic compounds of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof Besides inert diluents, the oral compositions can also include adjuvants such as wetting agents, emulsifying and suspending agents, sweetening, flavoring, coloring, perfuming, and preservative agents.

In various embodiments, topical administration of the pharmaceutical compositions, either to skin or to mucosal membranes, is contemplated. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface-active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the disclosure (e.g., a bifunctional antagonist molecule), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Additional pharmaceutical compositions contemplated for use herein include formulations involving polypeptides in sustained- or controlled-delivery formulations. Techniques for formulating a variety of other sustained- or controlled-delivery means, such as liposome carriers, bio-erodible microparticles or porous beads and depot injections, are also known to those skilled in the art.

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the polypeptide is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. Polypeptide compositions may be preferably injected or administered intravenously. Long-acting pharmaceutical compositions may be administered every three to four days, every week, or biweekly depending on the half-life and clearance rate of the particular formulation. The frequency of dosing will depend upon the pharmacokinetic parameters of the polypeptide in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g., orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, or intraperitoneal; as well as intranasal, enteral, topical, sublingual, urethral, vaginal, or rectal means, by sustained release systems or by implantation devices. Where desired, the compositions may be administered by bolus injection or continuously by infusion, or by implantation device. Alternatively, or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

### Therapeutic Uses

In another aspect, the present disclosure provides a method of treating or preventing various complex disease conditions whose pathogenesis involve the activation of both RANKL-NFκB and TGFβ/Activin-mediated Smad2/3 signaling pathway.

In various embodiments, the novel bifunctional antagonist molecules of the present invention may have broad applications for the treatment of various disorders which include, but are not limited to, the following conditions: Metastasis: bone metastasis, lung metastasis, liver metastasis, and brain metastasis; Bone cancers: multiple myeloma, osteosarcoma, chondrosarcoma and Ewing's sarcoma; Bone disorders: osteolytic lesions and skeletal-related event in cancer, bone fragility, osteogenesis imperfecta, fracture, osteopenia, and osteoporosis.

The present disclosure provides methods of treating bone disease in a subject, comprising administering a therapeutically effective amount of the pharmaceutical compositions of the invention to a subject in need thereof. In one embodiment, the subject is a human subject. In various embodiments, the bone disease is selected from osteomalacia, osteoporosis, osteogenesis imperfecta, fibrodysplasia ossificans progressive, corticosteroid-induced bone loss, bone loss associated with androgen-deprivation therapy, bone fracture, bone loss in cancer, bone metastasis and osteolytic lesions.

The present disclosure provides for a method of inhibiting loss of muscle mass and/or muscle function in a subject comprising administering an effective amount of a bifunctional antagonist molecule into the subject. The modulation may attenuate the loss of the muscle mass and/or function of said subject by at least 5%, 10%, at least 25%, at least 50%, at least 75%, or at least 90%. The inhibition of loss of muscle mass and function can be evaluated by using imaging techniques and physical strength tests. Examples of imaging techniques for muscle mass evaluation include Dual-Energy X-Ray Absorptiometry (DEXA), Magnetic Resonance Imaging (MRI), and Computed Tomography (CT). Examples of muscle function tests include grip strength test, stair climbing test, short physical performance battery (SPPB) and 6-minute walk, as well as maximal inspiratory pressure (MIP) and maximal expiratory pressure (MEP) that are used to measure respiratory muscle strength.

The present disclosure provides for a method of treating cancer cells as well as cancer cell metastasis in a subject, comprising administering to said subject a therapeutically effective amount (either as monotherapy or in a combination therapy regimen) of a bifunctional antagonist molecule of the present disclosure in pharmaceutically acceptable carrier, wherein such administration inhibits the growth and/or proliferation of a cancer cell. Specifically, a bifunctional antagonist molecule of the present disclosure is useful in treating disorders characterized as cancer. Such disorders include, but are not limited to solid tumors, such as cancers of the breast, respiratory tract, brain, reproductive organs, digestive tract, urinary tract, eye, liver, skin, head and neck, thyroid, parathyroid and their distant metastases, lymphomas, sarcomas, multiple myeloma and leukemia. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to, small-cell and non-small-cell lung carcinoma, as well as bronchial adenoma and pleuropulmonary blastoma. Examples of brain cancers include, but are not limited to, brain stem and hypophthalmic glioma, cerebellar and cerebral astrocytoma, medulloblastoma, ependymoma, as well as neuroectodermal and pineal tumor. Tumors of the male reproductive organs include, but are not limited to, prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, esophageal, gallbladder, gastric, pancreatic, rectal, small-intestine, and salivary gland cancers. Tumors of the urinary tract include, but are not limited to bladder, penile, kidney, renal pelvis, ureter, and urethral cancers. Eye cancers include, but are not limited to, intraocular melanoma and retinoblastoma. Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to nasopharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system. Sarcomas include, but are not limited to, sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia. In certain embodiments, the cancer will be a cancer with high expression of TNF-α and TGF-β, e.g., pancreatic cancer, gastric cancer, ovarian cancer, colorectal cancer, melanoma, leukemia, lung cancer, prostate cancer, brain cancer, bladder cancer, and head-neck cancer.

“Therapeutically effective amount” or “therapeutically effective dose” refers to that amount of the therapeutic agent being administered which will relieve to some extent one or more of the symptoms of the disorder being treated.

A therapeutically effective dose can be estimated initially from cell culture assays by determining an IC₅₀. A dose can then be formulated in animal models to achieve a circulating plasma concentration range that includes the 1050 as determined in cell culture. Such information can be used to more accurately determine useful doses in humans. Levels in plasma may be measured, for example, by HPLC. The exact composition, route of administration and dosage can be chosen by the individual physician in view of the subject's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

An exemplary, non-limiting daily dosing range for a therapeutically or prophylactically effective amount of a bifunctional antagonist molecule of the disclosure can be 0.001 to 100 mg/kg, 0.001 to 90 mg/kg, 0.001 to 80 mg/kg, 0.001 to 70 mg/kg, 0.001 to 60 mg/kg, 0.001 to 50 mg/kg, 0.001 to 40 mg/kg, 0.001 to 30 mg/kg, 0.001 to 20 mg/kg, 0.001 to 10 mg/kg, 0.001 to 5 mg/kg, 0.001 to 4 mg/kg, 0.001 to 3 mg/kg, 0.001 to 2 mg/kg, 0.001 to 1 mg/kg, 0.010 to 50 mg/kg, 0.010 to 40 mg/kg, 0.010 to 30 mg/kg, 0.010 to 20 mg/kg, 0.010 to 10 mg/kg, 0.010 to 5 mg/kg, 0.010 to 4 mg/kg, 0.010 to 3 mg/kg, 0.010 to 2 mg/kg, 0.010 to 1 mg/kg, 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.1 to 4 mg/kg, 0.1 to 3 mg/kg, 0.1 to 2 mg/kg, 0.1 to 1 mg/kg, 1 to 50 mg/kg, 1 to 40 mg/kg, 1 to 30 mg/kg, 1 to 20 mg/kg, 1 to 10 mg/kg, 1 to 5 mg/kg, 1 to 4 mg/kg, 1 to 3 mg/kg, 1 to 2 mg/kg, or 1 to 1 mg/kg body weight. It is to be noted that dosage values may vary with the type and severity of the conditions to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In various embodiments, the total dose administered will achieve a plasma antibody concentration in the range of, e.g., about 1 to 1000 μg/ml, about 1 to 750 μg/ml, about 1 to 500 μg/ml, about 1 to 250 μg/ml, about 10 to 1000 μg/ml, about 10 to 750 μg/ml, about 10 to 500 μg/ml, about 10 to 250 μg/ml, about 20 to 1000 μg/ml, about 20 to 750 μg/ml, about 20 to 500 μg/ml, about 20 to 250 μg/ml, about 30 to 1000 μg/ml, about 30 to 750 μg/ml, about 30 to 500 μg/ml, about 30 to 250 μg/ml.

Toxicity and therapeutic index of the pharmaceutical compositions of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

The dosing frequency of the administration of the bifunctional antagonist molecule pharmaceutical composition depends on the nature of the therapy and the particular disease being treated. The subject can be treated at regular intervals, such as weekly or monthly, until a desired therapeutic result is achieved. Exemplary dosing frequencies include, but are not limited to: once weekly without break; once weekly, every other week; once every 2 weeks; once every 3 weeks; weakly without break for 2 weeks, then monthly; weakly without break for 3 weeks, then monthly; monthly; once every other month; once every three months; once every four months; once every five months; or once every six months, or yearly.

### Combination Therapy

As used herein, the terms “co-administration”, “co-administered” and “in combination with”, referring to the a bifunctional antagonist molecule of the disclosure and one or more other therapeutic agents, is intended to mean, and does refer to and include the following: simultaneous administration of such combination of a bifunctional antagonist molecule of the disclosure and therapeutic agent(s) to a subject in need of treatment, when such components are formulated together into a single dosage form which releases said components at substantially the same time to said subject; substantially simultaneous administration of such combination of a bifunctional antagonist molecule of the disclosure and therapeutic agent(s) to a subject in need of treatment, when such components are formulated apart from each other into separate dosage forms which are taken at substantially the same time by said subject, whereupon said components are released at substantially the same time to said subject; sequential administration of such combination of a bifunctional antagonist molecule of the disclosure and therapeutic agent(s) to a subject in need of treatment, when such components are formulated apart from each other into separate dosage forms which are taken at consecutive times by said subject with a significant time interval between each administration, whereupon said components are released at substantially different times to said subject; and sequential administration of such combination of a bifunctional antagonist molecule of the disclosure and therapeutic agent(s) to a subject in need of treatment, when such components are formulated together into a single dosage form which releases said components in a controlled manner whereupon they are concurrently, consecutively, and/or overlappingly released at the same and/or different times to said subject, where each part may be administered by either the same or a different route.

In another aspect, the present disclosure provides a method for treating cancer or cancer metastasis in a subject, comprising administering a therapeutically effective amount of the pharmaceutical compositions of the invention in combination with a second therapy selected from the group consisting of: cytotoxic chemotherapy, immunotherapy, small molecule kinase inhibitor targeted therapy, surgery, radiation therapy, and stem cell transplantation. In various embodiments, the combination therapy may comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, RANKL, PD-L1, OX-40, CD137, TIGIT, GITR, LAG3, TIM-3, CD47, SIRPα, ICOS, and VISTA; treatment using bispecific T cell engaging antibodies (BiTE®) such as blinatumomab: treatment involving administration of biological response modifiers such as TNF family, IL-1, IL-4, IL-7, IL-12, IL-15, IL-17, IL-21, IL-22, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR: TLR7, TLR8, and TLR 9) agonists CpG and imiquimod; wherein the combination therapy provides increased effector cell killing of tumor cells, i.e., a synergy exists between the bifunctional antagonists molecule antagonist molecule and the immunotherapy when co-administered.

In various embodiments, the combination therapy comprises administering a bifunctional antagonist molecule and the second agent composition simultaneously, either in the same pharmaceutical composition or in separate pharmaceutical composition. In various embodiments, a bifunctional antagonist molecule composition and the second agent composition are administered sequentially, i.e., a bifunctional antagonist molecule composition is administered either prior to or after the administration of the second agent composition.

In various embodiments, the administrations of a bifunctional antagonist molecule composition and the second agent composition are concurrent, i.e., the administration period of a bifunctional antagonist molecule composition and the second agent composition overlap with each other.

In various embodiments, the administrations of a bifunctional antagonist molecule composition and the second agent composition are non-concurrent. For example, in various embodiments, the administration of a bifunctional antagonist molecule composition is terminated before the second agent composition is administered. In various embodiments, the administration second agent composition is terminated before a bifunctional antagonist molecule composition is administered.

The following examples are offered to more fully illustrate the disclosure but are not construed as limiting the scope thereof.

### EXAMPLE 1

The bifunctional antagonist molecules of the present disclosure can be prepared according to recombinant DNA techniques that are well known to those of skill in the art. In this example, the preparation of the bifunctional antagonist molecules is generally described.

cDNAs encoding various novel bifunctional antagonist polypeptides were generated via gene synthesis and subcloned into mammalian expression plasmids. CHO cells were transiently or stably transfected with the mammalian expression plasmids encoding the individual bifunctional polypeptide antagonists. Transiently transfected CHO cells or stably transfected CHO pools were grown in high-density suspension cultures in a CO₂ shaking incubator at 32° C. for 5-8 days. The culture media were collected after passing through a 0.22 μm filter unit (Millipore Corporation, MA). The recombinantly expressed bifunctional polypeptides were purified from the culture media via Protein A affinity chromatography using an AKTA PFLC system (GE Healthcare).

### EXAMPLE 2

Binding activities of individual bifunctional antagonists to human ligands or target proteins were measured by biolayer interferometry (BLI) using Octet RED96 (FortéBIO, Pall Corporation, USA). Binding analysis was performed by first capturing the polypeptide bifunctional antagonists to biosensors followed by two baseline steps in 1× kinetic buffer. The bifunctional antagonists-captured biosensors were then submerged in wells containing different concentrations of specific individual ligand, such as RANKL, Activin A, activin B, Activin AB, myostatin, TGF-β1, TGF-β2 or TGF-β3, for 6 min association followed by 6 min of dissociation time in 1× kinetic buffer. The bifunctional antagonists-captured sensors were also dipped in wells containing 1× kinetic buffer to allow single reference subtraction in order to compensate for the natural dissociation of captured bifunctional antagonists. The binding sensorgrams were collected using the 8-channel detection mode on the Octet RED96 biosensor. Data were acquired and analyzed using the data acquisition software v11.1 (FortéBIO, Pall Corporation, USA).

The binding affinities of bifunctional antagonist molecules A112, A113 and A114 were examined using BLI analysis. As depicted in Table 9, bifunctional polypeptide antagonists A112, A113 and A114 were able to selectively bind to RANKL, Activin A, Activin B, Activin AB and myostatin with high affinity.

TABLE 9
Binding Affinity KD (M)

| | RANKL | Activin A | Activin B | Activin AB | Myostatin |
|---|---|---|---|---|---|
| A112 | ~1.0E−12 | ~6.0E−11 | ~2.0E−10 | ~3.0E−10 | ~1.0E−12 |
| A113 | ~8.0E−11 | ~3.0E−11 | ~1.0E−12 | ~1.0E−12 | ~1.0E−12 |
| A114 | ~1.0E−12 | ~5.0E−11 | ~1.0E−10 | ~6.0E−11 | ~1.0E−12 |

The binding affinities of bifunctional antagonist molecules A239 and A240 were examined using BLI analysis. As shown in Table 10, A239 and A240 are able to bind RANKL, TGF-β1 and TGF-β3 with high affinity.

TABLE 10
Binding Affinity KD (M)

| | RANKL | TGF-β1 | TGF-β2 | TGF-β3 |
|---|---|---|---|---|
| A239 | ~4.0E−11 | ~5.0E−12 | ~1.0E−6 | ~3.0E−11 |
| A240 | ~1.0E−11 | ~6.0E−12 | ~5.0E−7 | ~1.0E−11 |

The ligand binding affinities of A237 and A241, two exemplary bispecific antibodies of the present invention, were examined using BLI analysis. As shown in Table 11, A237 binds RANKL and activin A with high affinity (Table 11A), whereas A241 binds RANKL and TGF-β with high affinity (Table 11B).

TABLE 11
A.

| | Activin A Binding | RANKL Binding |
|---|---|---|
| | KD (M) | KD (M) |
| A237 | ~3.0E−10 | ~1.0E−12 |

B.

| | RANKL Binding | TGF-β1 Binding | TGF-β3 Binding |
|---|---|---|---|
| | KD (M) | KD (M) | KD (M) |
| A241 | ~7.0E−10 | ~2.0E−09 | ~3.0E−10 |

### EXAMPLE 3

Smad2/3 signaling assay. An engineered luciferase reporter cell line, C2C12-CAGA-luc, capable of sensing Smad2/3 signaling was used to measure activin A, activin B, Activin AB, myostatin and GDF-11 signaling activities in cell cultures. To measure neutralizing activities of bifunctional antagonists, 2 nM of human ligand (i.e., human activin A, activin B, Activin AB, myostatin, and GDF-11, respectively) was preincubated with increasing concentrations of each bifunctional antagonist at 0.00004 nM, 0.0004 nM, 0.004 nM, 0.04 nM, 0.4 nM, 4 nM, 40 nM and 400 nM for 1 hour at room temperature. Subsequently, the reaction mixtures were added to the C2C12-CAGA-luc cell cultures. After incubation for 5 hours in CO2 incubator at 37° C., the luciferase activities of the C2C12-CAGA-luc reporter cultures were measured by using LuminoSkan Ascent (Thermo Scientific). The IC₅₀ values were analyzed and plotted using Prism software (GraphPad Software).

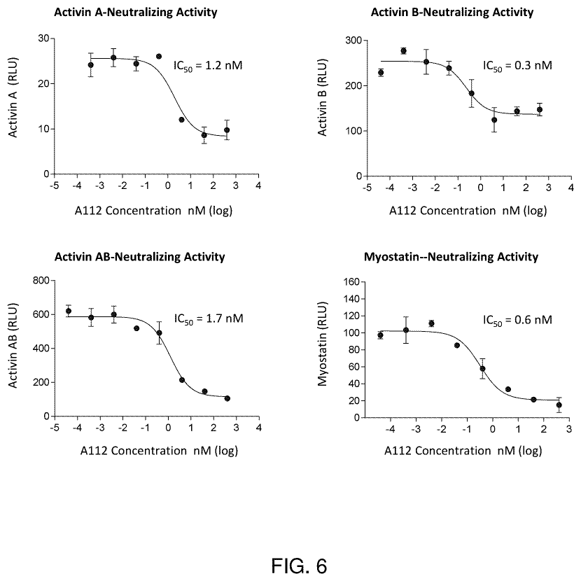

As depicted in FIG. 6, bifunctional antagonist A112 strongly neutralize Activin A, Activin B, Activin AB and myostatin in cell-based assays.

### EXAMPLE 4

A112, a representative bifunctional antagonist capable of simultaneously sequestering RANKL and activin was evaluated in comparison to anti-RANKL antibody and ActRIIA-Fc, respectively, for its ability to inhibit osteoclastogenesis mediated by RANKL and activin A in RAW 246.7 cells. In addition, A240, a representative bifunctional antagonist capable of simultaneously neutralizing RANKL and TGF-β was examined in comparison to anti-RANKL antibody and TGFRII-Fc, respectively, for its ability to suppress osteoclastogenesis induced by RANKL and TGF-β in RAW 246.7 cells.

RAW 246.7 cell-based osteoclast formation assay. RAW 246.7 cells were grown in RPMI-1640 medium supplemented with 10% FBS in 6-well culture plates and were maintained in a CO2 incubator at 37° C. To evaluate the effect of A112, RAW 246.7 cultures were incubated in the presence or absence of exogenously added agents as follows: (1) (1) No addition (control), (2) 100 ng/mL RANKL, (3) 100 ng/mL activin A, (4) 100 ng/mL RANKL and 100 ng/mL activin A, (5) 100 ng/mL RANKL, 100 ng/mL activin A and 2.5 ug/mL anti-RANKL antibody, (6) 100 ng/mL RANKL, 100 ng/mL activin A and 2.5 ug/mL TGFRII-Fc, and (7) 100 ng/mL RANKL, 100 ng/mL activin A and 2.5 ug/mL A112. To evaluate the effect of A240, RAW 246.7 cultures were incubated in the presence or absence of various exogenously added agents as follows: (1) No addition (control), (2) 100 ng/mL RANKL, (3) 5 ng/mL TGF-β1, (4) 100 ng/mL RANKL and 5 ng/mL TGF-β1, (5) 100 ng/mL RANKL, 5 ng/mL TGF-β1 and 2.5 ug/mL anti-RANKL antibody, (6) 100 ng/mL RANKL, 5 ng/mL TGF-β1 and 2.5 ug/mL TGFRII-Fc, and (7) 100 ng/mL RANKL, 5 ng/mL TGF-β1 and 2.5 ug/mL A240. After five days of incubation under the different conditions, the RAW 246.7 cultures were subjected to TRAP staining using a trap staning kit (Sigma) by following the Manufacturer's instructions. Osteoclast formation defined as TRAP staning-positve multinucleated cells containg 3 or more cell nuclei and the number of osteoclasts were counted. Representative osteoclast formation images were photographed with a inverted microscope quipped with digital camera.

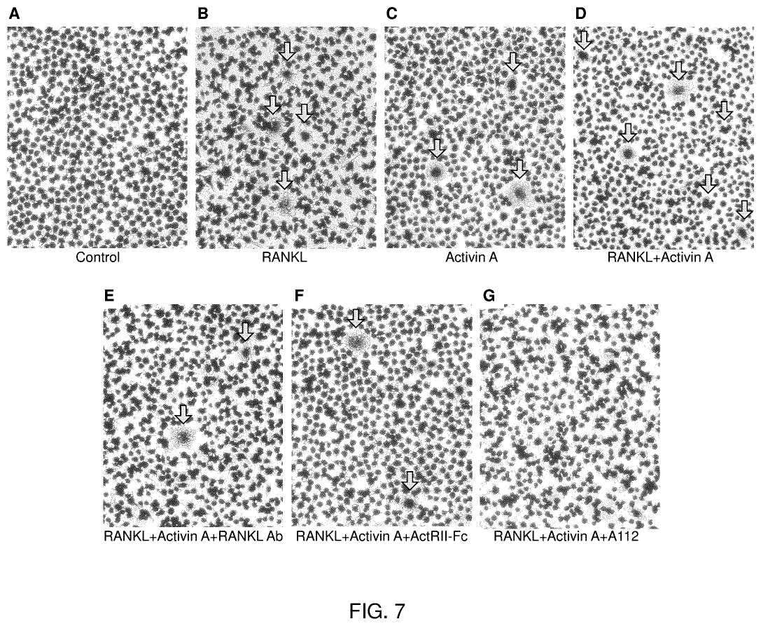

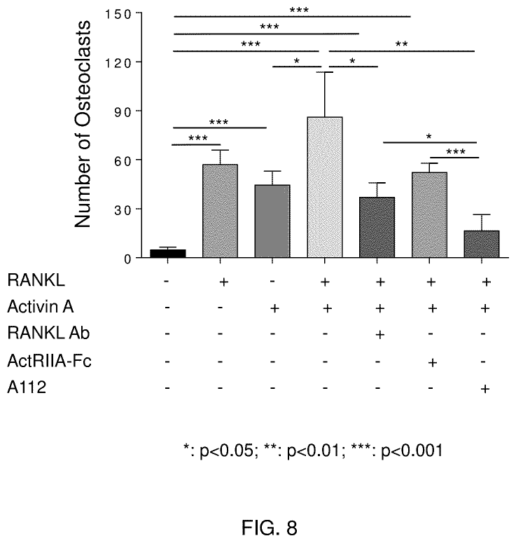

FIG. 7 depicts the photograph images of the control RAW 246.7 cell culture and RAW 246.7 cell cultures treated with different conditions with arrows pointing to the osteoclast formations. Under control condition, no osteoclast formation was detected in the RAW 246.7 culture (FIG. 7, panel A). After 5-day treatment with RANKL or activin A, osteoclasts were detected in the RAW 246.7 cultures as TRAP-positive multinucleated cells (FIG. 7, panels B and C). After 5-day co-treatment with RANKL and activin A, multinucleated TRAP-positive cells were detected in higher density in the RAW 246.7 culture (FIG. 7, panel D), suggesting an enhanced osteoclastogenesis of RAW 246.7 cells in response to elevated RANKL and activin A. In the presence of exogenously added RANKL and activin A, treatment with RANKL antibody, ActRIIA-Fc and A112, respectively, all reduced the density of osteoclast formation in the RAW 246.7 cultures. Quantitative morphometric analysis of the RAW 246.7 cell cultures (FIG. 8) revealed that compare to the RANKL antibody-treated culture or the ActRIIA-Fc-treated culture, the A112-treated culture showed a significantly greater reduction in osteoclast formation. Thus, the data suggest that A112 was more effective than either anti-RANKL antibody or ActRIIA-Fc in preventing the RANKL- and activin A-induced osteoblastogenesis in RAW 246.7 cells.

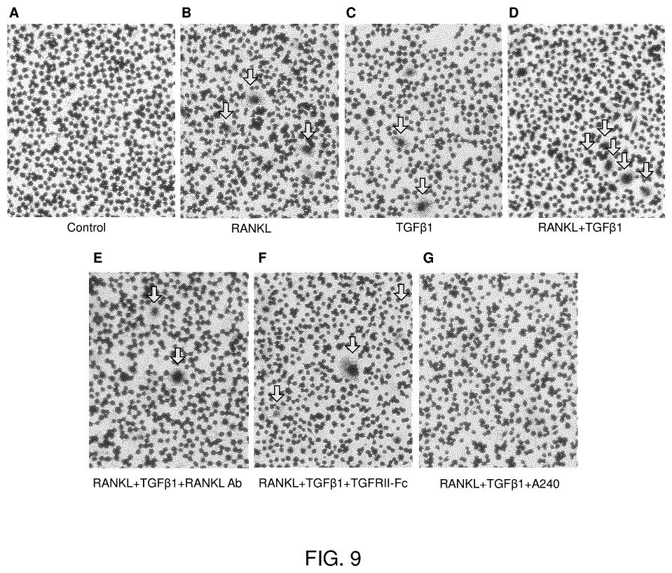

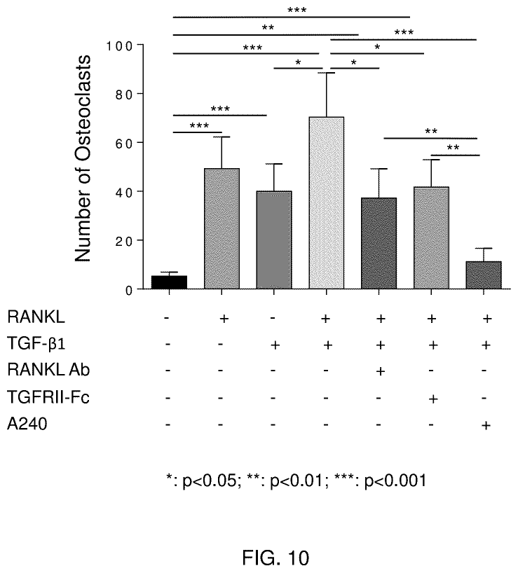

FIG. 9 depicts the photograph images of the control RAW 246.7 cell culture and RAW 246.7 cell cultures treated with different conditions with arrows pointing to the osteoclast formations. Under control condition, no osteoclast formation was detected in the RAW 246.7 culture (FIG. 9, panel A). After 5-day treatment with RANKL or TGF-β1, osteoclasts were appeared in the RAW 246.7 cultures as TRAP-positive multinucleated cells (FIG. 9, panels B and C). After 5-day co-treatment with RANKL and TGF-β1, multinucleated TRAP-positive cells appeared in higher density in the RAW 246.7 culture (FIG. 9, panel D), suggesting an enhanced osteoclastogenesis of RAW 246.7 cells in response to elevated RANKL and TGF-β1. In the presence of exogenously added RANKL and TGF-β1, treatment with RANKL antibody, TGFRII-Fc and A240, respectively, all decreased the density of osteoclast formation in the RAW 246.7 cultures. Quantitative morphometric analysis of the RAW 246.7 cell cultures (FIG. 10) revealed that compare to the RANKL antibody-treated culture or the TGFRII-Fc-treated culture, the A240-treated culture showed a significantly greater reduction in osteoclast formation. Thus, compared to anti-RANKL antibody or TGFRII-Fc, A240 was able to more effectively prevent the RANKL-and activin A-induced osteoblastogenesis in RAW 246.7 cells.

### EXAMPLE 5

A mouse model of glucocorticoid-induced bone loss was used to evaluate the effectiveness of parallel inhibition of activin and RANKL in preventing bone loss. In this mouse study, co-admisnistration of ActRIIA-Fc and anti-murine antibody was used as a surrogate treatment to mimic the action of bifunctional antagonist A112. It is important note that due to the lack of high homology between human RANKL and rodent RANKL, the bifunctional anatgonists of the present invention designed to bind human RANKL do not interact with the endogenous RANKL in rodents. To evaluate the in vivo pharmacological effect of RANKL inhibition in mice, it is necessary to use a RANKL inhibitor capable of recognizing the endogenous murine RANKL. Accordingly, in this mouse experiment, a combination treatment with anti-murine RANKL antibody and ActRIIA-Fc was used as a surrogate treatment to mimic the pharmacological action of the bifunctional antagonist to simultenously sequester RANKL and activin in mice.

Experimental methods for studying mice with glucocorticoid-induced bone loss. Anti-murine RANKL antibody was purchased from BioxCell. Recombinant ActRIIA-Fc with >98% purity was purified from medium of transiently transfected CHO cultures with protein A chromatography followed by size exclusion chromatography. Ten-week-old female CD1 mice were purchased from Envigo. The mice were fed with dexamethasone (DEX) via drinking water at a dose of 2.4 mg/kg/day (DEX-fed mice). The DEX-fed mice were divided into four treatment groups (n=8/9 per group), which were administered, respectively, the following agents via subcutaneous injection: (1) PBS (vehicle), (2) ActRIIA-Fc (10 mg/kg), (3) Anti-murine RANKL antibody (10mg/kg), and (4) ActRIIA-Fc (10 mg/kg) and anti-murine RANKL antibody (10 mg/kg) in combination. The DEX-fed mice were treated once per week for a period of 4 weeks. Aged-matched female CD1 mice that did not receive dexamethasone (DEX) in the drinking water served as the control group (n=8). At the end of the experiment, all the animals were euthanized by carbon dioxide inhalation and the right femurs were dissected for imaging by microCT. For the bone imaging analysis, Skyscan 1172 microCT Instrument and the CTrecon and CTan softaware from Skyscan were used. Briefly, femurs were placed in a Styrofoam mold fitted to the rotating stage in the machine. Bones were scanned, and the collection of images was reconstructed for analysis. 3D and 2D microCT imaging analysis were conducted on the trabecular and cortical bones, respectively, to obtain the parameters on bone volume and density.

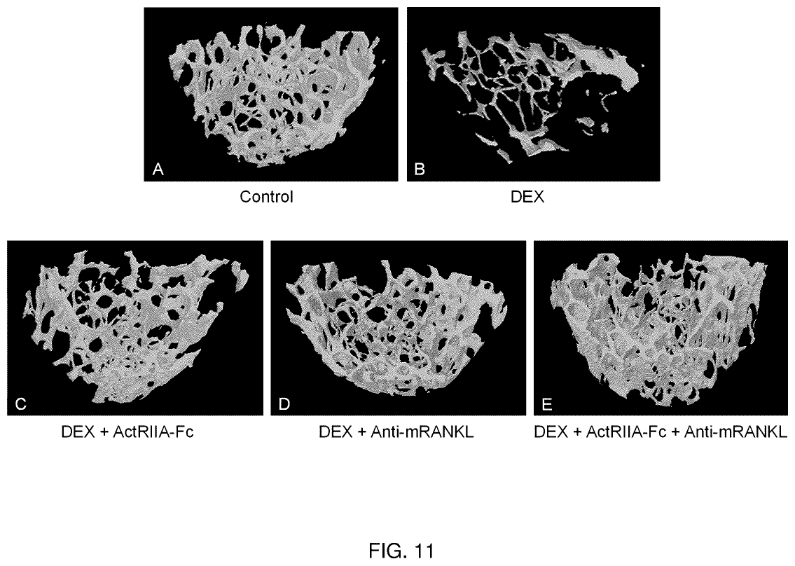

FIG. 11 shows the representative microCT images of bone volume of the distal femurs in the different mouse groups: (A) Control, (B) DEX (Dexamethasome fed), (C) DEX plus ActRIIA-Fc treatment, (D) DEX plus anti-murine RANKL antibody treatment, and (E) DEX plus the combination treatment with ActRIIA-Fc and anti-murine RANKL antibody. Note the marked bone loss resulting from dexamethasone (panel B vs. panel A). Treatment with ActRIIA-Fc, anti-murine RANKL antibody, or the combination of ActRIIA-Fc and anti-murine RANKL antibody, respectively, attenuated the glucocorticoid-induced bone loss. The combination treatment appeared to be more effective as it completed reversed the glucocorticoid-induced bone loss (FIG. 11, panel E).

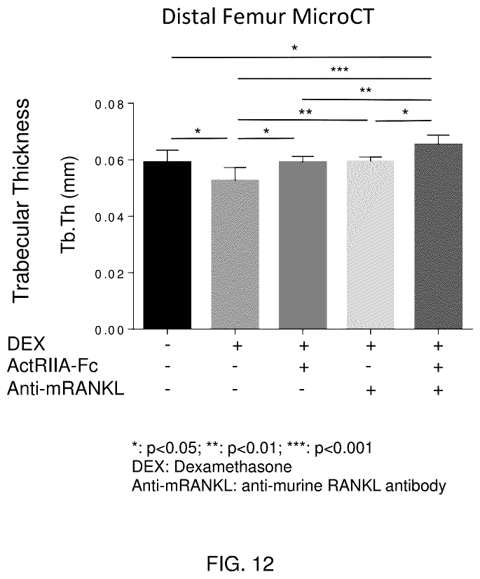

FIG. 12 shows the microCT imaging analysis on the average trabecular bone thickness of the distal femurs in the different mouse groups as illustrated in the figure. The data indicate that compared to normal control, there was a dramatic bone loss in the dexamethasome-fed mice. Treatment of the dexamethasome-fed mice with ActRIIA-Fc or anti-murine RANKL antibody significantly attenuated bone loss. The combination treatment with with ActRIIA-Fc and anti-murine RANKL antibody more effectively compared to treatment with ActRIIA-Fc alone or anti-murine RANKL antibody alone, as the combination treatment not only fully ameliorated bone loss but its effect in countering bone loss was statistically greater than that of ActRIIA-Fc or anti-murine RANKL antibody. The data suggest that the combination treatment enhanced the bone mass in the dexamethasome-fed mice to a level that was statistically greater than normal control.

Taken together, the data presented in this example demonstrated that as a surrogate treatment for bifunctional antagonist A112, the combined administration of ActRIIA-Fc and anti-murine RANKL antibody was able to reverse the glucocorticoid-induced bone loss in the dexamethasone-fed mice more effectively than administration of ActRIIA-Fc alone or anti-murine RANKL antibody alone. The stronger effect in counteracting bone loss seen upon parallel inhibition of activin and RANKL by the combination treatment with ActRIIA-Fc and anti-murine RANKL in mice suggest that bifunctional antagonist A112 as well as other novel bifunctional antagonists of activin and RANKL as disclosed in the present invention may represent a more efficacious approach to treating bone loss and fragility conditions in human patients.

All of the articles and methods disclosed and claimed herein can be made and executed without undue experimentation in light of the present invention. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### Sequence Listings

The nucleic and amino acid sequences listed in the accompanying sequence listing are shown using standard letter abbreviations for nucleotide bases and three letter code for amino acids, as defined in 37 C.F.R. 1.822.
SEQ ID NOs: 1-9 are amino acid sequences of various Activin or Activin-related ligands.
SEQ ID NOs: 10 and 14 are amino acid sequences of a heavy chain of various antibodies which specifically binds to Activin or Activin-related ligand.
SEQ ID NOs: 11 and 15 are amino acid sequences of a light chain of various antibodies which specifically binds to Activin or Activin-related ligand.
SEQ ID NOs: 12 and 16 are amino acid sequences of a heavy chain variable region of various antibodies which specifically binds to Activin or Activin-related ligand.
SEQ ID NOs: 13 and 17 are amino acid sequences of a light chain variable region of various antibodies which specifically binds to Activin or Activin-related ligand.
SEQ ID NOs: 18-19 are amino acid sequences of various TGF-β ligands.
SEQ ID NO: 20 is an amino acid sequence of a heavy chain of an antibody which specifically binds to TGF-β ligand.
SEQ ID NO: 21 is an amino acid sequences of a light chain of an antibody which specifically binds to TGF-β ligand.
SEQ ID NO: 22 is an amino acid sequence of a heavy chain variable region of an antibody which specifically binds to TGF-β ligand.
SEQ ID NO: 23 is an amino acid sequences of a light chain variable region of an antibody which specifically binds to TGF-β ligand.
SEQ ID NO: 24 is an amino acid sequence of a heavy chain of an antibody which specifically binds to RANKL ligand.
SEQ ID NO: 25 is an amino acid sequences of a light chain of an antibody which specifically binds to RANKL ligand.
SEQ ID NO: 26 is an amino acid sequence of a heavy chain variable region of an antibody which specifically binds to RANKL ligand.
SEQ ID NO: 27 is an amino acid sequences of a light chain variable region of an antibody which specifically binds to RANKL ligand.
SEQ ID NO: 28 is an amino acid sequence of Human Osteoprotegerin (OPG).
SEQ ID NOs: 29-37 are the amino acid sequences of a heavy chain of a bifunctional antagonist molecule that specifically binds to Activin or Activin-related ligand and RANKL.
SEQ ID NOs: 38-39 are the amino acid sequences of a heavy chain of a bifunctional antagonist molecule that specifically binds to TGF-β ligand and RANKL.
SEQ ID NOs: 40-48 are the amino acid sequences of various bifunctional antagonist molecules that specifically binds to Activin or Activin-related ligand and that specifically binds to OPG.
SEQ ID NOs: 49-50 are the amino acid sequences of a heavy chain of a bifunctional antagonist molecule that specifically binds to Activin or Activin-related ligand and OPG.
SEQ ID NOs: 51-52 are the amino acid sequences of various bifunctional antagonist molecules that specifically binds to TGF-β ligand and that specifically binds to OPG.
SEQ ID NO: 53 is the amino acid sequences of a heavy chain of a bifunctional antagonist molecule that specifically binds to TGF-β ligand and OPG.
SEQ ID NO: 54 is the amino acid sequence of a heavy chain of a bifunctional antagonist molecule that specifically binds to Activin or Activin-related ligand and RANKL.
SEQ ID NO: 55 is the amino acid sequence of a light chain of a bifunctional antagonist molecule that specifically binds to Activin or Activin-related ligand and RANKL.
SEQ ID NO: 56 is the amino acid sequence of a heavy chain of a bifunctional antagonist molecule that specifically binds to Activin or Activin-related ligand and RANKL.
SEQ ID NO: 57 is the amino acid sequence of a light chain of a bifunctional antagonist molecule that specifically binds to Activin or Activin-related ligand and RANKL.
SEQ ID NO: 58 is the amino acid sequence of a heavy chain of a bifunctional antagonist molecule that specifically binds to TGF-β ligand and RANKL.
SEQ ID NO: 59 is the amino acid sequence of a light chain of a bifunctional antagonist molecule that specifically binds to TGF-β ligand and RANKL.
SEQ ID NOs: 60-79 are the amino acid sequences of various peptide linker sequences.

SEQUENCE LISTINGS

Human ActRIIA-ECD
ETQECLFFNANWEKDRTNQTGVEPCYGDKDKRRHCFATWKNISGSIEIVKQGCWLDDINCYD
RTDCVEKKDSPEVYFCCCEGNMCNEKFSYFPEMEVTQPTSNPVTPKPP (SEQ ID NO: 1)
Human ActRIIB-ECD
ETRECIYYNANWELERTNQSGLERCEGEQDKRLHCYASWRNSSGTIELVKKGCWLDDFNCYD
RQECVATEENPQVYFCCCEGNFCNERFTHLPEAGGPEVTYEPPPTAPT (SEQ ID NO: 2)
Human Follistatin 315
GNCWLRQAKNGRCQVLYKTELSKEECCSTGRLSTSWTEEDVNDNTLFKWMIFNGGAPNCIPC
KETCENVDCGPGKKCRMNKKNKPRCVCAPDCSNITWKGPVCGLDGKTYRNECALLKARCKE
QPELEVQYQGRCKKTCRDVFCPGSSTCVVDQTNNAYCVTCNRICPEPASSEQYLCGNDGVTY
SSACHLRKATCLLGRSIGLAYEGKCIKAKSCEDIQCTGGKKCLWDFKVGRGRCSLCDELCPDS
KSDEPVCASDNATYASECAMKEAACSSGVLLEVKHSGSCNSISEDTEEEEEDEDQDYSFPISSI
LEW (SEQ ID NO: 3)
Human Follistatin ΔHBS (modified Follistatin)
GNCWLRQAKNGRCQVLYKTELSKEECCSTGRLSTSWTEEDVNDNTLFKWMIFNGGAPNCIPC
KETCENVDCGPGQSCVVDQTGSPRCVCAPDCSNITWKGPVCGLDGKTYRNECALLKARCKE
QPELEVQYQGRCKKTCRDVFCPGSSTCVVDQTNNAYCVTCNRICPEPASSEQYLCGNDGVTY
SSACHLRKATCLLGRSIGLAYEGKCIKAKSCEDIQCTGGKKCLWDFKVGRGRCSLCDELCPDS
KSDEPVCASDNATYASECAMKEAACSSGVLLEVKHSGSCNSISEDTEEEEEDEDQDYSFPISSI
LEW (SEQ ID NO: 4)
Human Follistatin 288
GNCWLRQAKNGRCQVLYKTELSKEECCSTGRLSTSWTEEDVNDNTLFKWMIFNGGAPNCIPC
KETCENVDCGPGKKCRMNKKNKPRCVCAPDCSNITWKGPVCGLDGKTYRNECALLKARCKE
QPELEVQYQGRCKKTCRDVFCPGSSTCVVDQTNNAYCVTCNRICPEPASSEQYLCGNDGVTY
SSACHLRKATCLLGRSIGLAYEGKCIKAKSCEDIQCTGGKKCLWDFKVGRGRCSLCDELCPDS
KSDEPVCASDNATYASECAMKEAACSSGVLLEVKHSGSCN (SEQ ID NO: 5)
Modified human ActRIIB ECD
ETRECIYYNANWELERTNQSGLERCEGDQDKRLHCYASWRNSSGTIELVKKGCWLDDINCYD
RQECVATKENPQVYFCCCEGNFCNERFTHLPEAGGPEVTYEPPPTAPT (SEQ ID NO: 6)
Modified human ActRIIB ECD
ETRECIYYNANWELERTNQSGLERCYGDKDKRRHCYASWRNSSGTIELVKKGCWLDDFNCYD
RQECVATEENPQVYFCCCEGNFCNERFTHLPEAGGPEVTYEPPPTAPT (SEQ ID NO: 7)
Modified human ActRIIB ECD
ETRECIYYNANWELERTNQSGLERCEGEQDKRLHCYASWRNSSGTIELVKKGCWDDDFNCY
DRQECVATEENPQVYFCCCEGNFCNERFTHLPEAGGPEVTYEPPPT (SEQ ID NO: 8)
Modified human ActRIIA ECD
GAILGRSETQECLFYNANWELERTNQTGVEPCEGEKDKRLHCYATWRNISGSIEIVKKGCWLD
DFNCYDRTDCVETEENPQVYFCCCEGNMCNEKFSYFPEMEVTQPTS (SEQ ID NO: 9)
Anti-Activin antibody heavy chain amino acid sequence
QVQLVQSGAEVKKPGASVKVSCKASGYTFTSYGLSWVRQAPGQGLEWMGWIIPYNGNTNSA
QKLQGRVTMTTDTSTSTAYMELRSLRSDDTAVYFCARDRDYGVNYDAFDIWGQGTMVTVSSA
STKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNVYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEALHN
HYTQKSLSLSPGK (SEQ ID NO: 10)
Anti-Activin A antibody light chain amino acid sequence
SYEVTQAPSVSVSPGQTASITCSGDKLGDKYACWYQQKPGQSPVLVIYQDSKRPSGIPERFSG
SNSGNTATLTISGTQAMDEADYYCQAWDSSTAVFGGGTKLTVLRTVAAPSVFIFPPSDEQLKS
GTASVVCLLNNFYPREAKVQWKVDNALQSGNSQESVTEQDSKDSTYSLSSTLTLSKADYEKH
KVYACEVTHQGLSSPVTKSFNRGEC (SEQ ID NO: 11)
Anti-Activin A antibody heavy chain variable region amino acid sequence
QVQLVQSGAEVKKPGASVKVSCKASGYTFTSYGLSWVRQAPGQGLEWMGWIIPYNGNTNSA
QKLQGRVTMTTDTSTSTAYMELRSLRSDDTAVYFCARDRDYGVNYDAFDIWGQGTMVTVSS
(SEQ ID NO: 12)
Anti-Activin A antibody light chain variable region amino acid sequence
SYEVTQAPSVSVSPGQTASITCSGDKLGDKYACWYQQKPGQSPVLVIYQDSKRPSGIPERFSG
SNSGNTATLTISGTQAMDEADYYCQAWDSSTAVFGGGTKLTVL (SEQ ID NO: 13)
Anti-Activin A antibody heavy chain amino acid sequence
QVQLQESGPGLVKPSETLSLTCTVSGGSFSSHFWSWIRQPPGKGLEWIGYILYTGGTSFNPSL
KSRVSMSVGTSKNQFSLKLSSVTAADTAVYYCARARSGITFTGIIVPGSFDIWGQGTMVTVSSA
STKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNH
YTQKSLSLSLGK (SEQ ID NO: 14)
Anti-Activin A antibody light chain amino acid sequence
EIVLTQSPGTLSLSPGERATLSCRASQSVSSSYLAWYQQKPGQAPRLLIYGASSRATGIPDRFS
GSGSGTDFTLTISRLEPEDFAVYYCQQYGSSPWTFGQGTKVEIKRTVAAPSVFIFPPSDEQLKS
GTASVVCLLNNFYPREAKVQWKVDNALQSGNSQESVTEQDSKDSTYSLSSTLTLSKADYEKH
KVYACEVTHQGLSSPVTKSFNRGEC (SEQ ID NO: 15)
Anti-Activin A antibody heavy chain variable region amino acid sequence
QVQLQESGPGLVKPSETLSLTCTVSGGSFSSHFWSWIRQPPGKGLEWIGYILYTGGTSFNPSL
KSRVSMSVGTSKNQFSLKLSSVTAADTAVYYCARARSGITFTGIIVPGSFDIWGQGTMVTVSS
(SEQ ID NO: 16)
Anti-Activin A antibody light chain variable region amino acid sequence
EIVLTQSPGTLSLSPGERATLSCRASQSVSSSYLAWYQQKPGQAPRLLIYGASSRATGIPDRFS
GSGSGTDFTLTISRLEPEDFAVYYCQQYGSSPWTFGQGTKVEIK (SEQ ID NO: 17)
Human TGF-β Receptor II-ECD isoform 1 (TGF-β RIIB-ECD)
TIPPHVQKSVNNDMIVTDNNGAVKFPQLCKFCDVRFSTCDNQKSCMSNCSITSICEKPQEVCV
AVWRKNDENITLETVCHDPKLPYHDFILEDAASPKCIMKEKKKPGETFFMCSCSSDECNDNIIFS
EEYNTSNPD (SEQ ID NO: 18)
Human TGF-β Receptor II-ECD isoform 2 (TGF-β RIIA-ECD)
TIPPHVQKSDVEMEAQKDEIICPSCNRTAHPLRHINNDMIVTDNNGAVKFPQLCKFCDVRFSTC
DNQKSCMSNCSITSICEKPQEVCVAVWRKNDENITLETVCHDPKLPYHDFILEDAASPKCIMKE
KKKPGETFFMCSCSSDECNDNIIFSEEYNTSNPD (SEQ ID NO: 19)
Anti-TGF-β antibody heavy chain amino acid sequence
QVQLVQSGAEVKKPGSSVKVSCKASGYTFSSNVISWVRQAPGQGLEWMGGVIPIVDIANYAQ
RFKGRVTITADESTSTTYMELSSLRSEDTAVYYCASTLGLVLDAMDYWGQGTLVTVSSASTKG
PSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPSCPAPEFLGGPSVFLFPPKPKDTL
MISRTPEVTCVVVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQ
KSLSLSLGK (SEQ ID NO: 20)
Anti-TGF-β antibody light chain amino acid sequence
ETVLTQSPGTLSLSPGERATLSCRASQSLGSSYLAWYQQKPGQAPRLLIYGASSRAPGIPDRF
SGSGSGTDFTLTISRLEPEDFAVYYCQQYADSPITFGQGTRLEIKRTVAAPSVFIFPPSDEQLKS
GTASVVCLLNNFYPREAKVQWKVDNALQSGNSQESVTEQDSKDSTYSLSSTLTLSKADYEKH
KVYACEVTHQGLSSPVTKSFNRGEC (SEQ ID NO: 21)
Anti-TGF-β antibody heavy chain variable region amino acid sequence
QVQLVQSGAEVKKPGSSVKVSCKASGYTFSSNVISWVRQAPGQGLEWMGGVIPIVDIANYAQ
RFKGRVTITADESTSTTYMELSSLRSEDTAVYYCASTLGLVLDAMDYWGQGTLVTVSS
(SEQ ID NO: 22)
Anti-TGF-β antibody light chain variable region amino acid sequence
ETVLTQSPGTLSLSPGERATLSCRASQSLGSSYLAWYQQKPGQAPRLLIYGASSRAPGIPDRF
SGSGSGTDFTLTISRLEPEDFAVYYCQQYADSPITFGQGTRLEIK (SEQ ID NO: 23)
Anti-RANKL antibody heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGK (SEQ ID NO: 24)
Anti-RANKL antibody light chain amino acid sequence
EIVLTQSPGTLSLSPGERATLSCRASQSVRGRYLAWYQQKPGQAPRLLIYGASSRATGIPDRFS
GSGSGTDFTLTISRLEPEDFAVFYCQQYGSSPRTFGQGTKVEIKRTVAAPSVFIFPPSDEQLKS
GTASVVCLLNNFYPREAKVQWKVDNALQSGNSQESVTEQDSKDSTYSLSSTLTLSKADYEKH
KVYACEVTHQGLSSPVTKSFNRGEC (SEQ ID NO: 25)
Anti-RANKL antibody heavy chain variable region amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSS
(SEQ ID NO: 26)
Anti-RANKL antibody light chain variable region amino acid sequence
EIVLTQSPGTLSLSPGERATLSCRASQSVRGRYLAWYQQKPGQAPRLLIYGASSRATGIPDRFS
GSGSGTDFTLTISRLEPEDFAVFYCQQYGSSPRTFGQGTKVEIK (SEQ ID NO: 27)
Human Osteoprotegerin (OPG) amino acid sequence
MNNLLCCALVFLDISIKWTTQETFPPKYLHYDEETSHQLLCDKCPPGTYLKQHCTAKWKTVCAP
CPDHYYTDSWHTSDECLYCSPVCKELQYVKQECNRTHNRVCECKEGRYLEIEFCLKHRSCPP
GFGVVQAGTPERNTVCKRCPDGFFSNETSSKAPCRKHTNCSVFGLLLTQKGNATHDNICSGN
SESTQKCGIDVTLCEEAFFRFAVPTKFTPNWLSVLVDNLPGTKVNAESVERIKRQHSSQEQTFQ
LLKLWKHQNKDQDIVKKIIQDIDLCENSVQRHIGHANLTFEQLRSLMESLPGKKVGAEDIEKTIKA
CKPSDQILKLLSLWRIKNGDQDTLKGLMHALKHSKTYHFPKTVTQSLKKTIRFLHSFTMYKLYQK
LFLEMIGNQVQSVKISCL (SEQ ID NO: 28)
A112 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSETQECLFFNANWEKDRTNQTGVEPCYGDKDKRRHCF
ATWKNISGSIEIVKQGCWLDDINCYDRTDCVEKKDSPEVYFCCCEGNMCNEKFSYFPEMEVTQ
PTSNPVTPKPP (SEQ ID NO: 29)
A114 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSETRECIYYNANWELERTNQSGLERCEGEQDKRLHCYA
SWRNSSGTIELVKKGCWLDDFNCYDRQECVATEENPQVYFCCCEGNFCNERFTHLPEAGGP
EVTYEPPPTAPT (SEQ ID NO: 30)
A231 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSGNCWLRQAKNGRCQVLYKTELSKEECCSTGRLSTSW
TEEDVNDNTLFKWMIFNGGAPNCIPCKETCENVDCGPGKKCRMNKKNKPRCVCAPDCSNITW
KGPVCGLDGKTYRNECALLKARCKEQPELEVQYQGRCKKTCRDVFCPGSSTCVVDQTNNAY
CVTCNRICPEPASSEQYLCGNDGVTYSSACHLRKATCLLGRSIGLAYEGKCIKAKSCEDIQCTG
GKKCLWDFKVGRGRCSLCDELCPDSKSDEPVCASDNATYASECAMKEAACSSGVLLEVKHS
GSCNSISEDTEEEEEDEDQDYSFPISSILEW (SEQ ID NO: 31)
A113 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSGNCWLRQAKNGRCQVLYKTELSKEECCSTGRLSTSW
TEEDVNDNTLFKWMIFNGGAPNCIPCKETCENVDCGPGQSCVVDQTGSPRCVCAPDCSNITW
KGPVCGLDGKTYRNECALLKARCKEQPELEVQYQGRCKKTCRDVFCPGSSTCVVDQTNNAY
CVTCNRICPEPASSEQYLCGNDGVTYSSACHLRKATCLLGRSIGLAYEGKCIKAKSCEDIQCTG
GKKCLWDFKVGRGRCSLCDELCPDSKSDEPVCASDNATYASECAMKEAACSSGVLLEVKHS
GSCNSISEDTEEEEEDEDQDYSFPISSILEW (SEQ ID NO: 32)
A232 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSGNCWLRQAKNGRCQVLYKTELSKEECCSTGRLSTSW
TEEDVNDNTLFKWMIFNGGAPNCIPCKETCENVDCGPGKKCRMNKKNKPRCVCAPDCSNITW
KGPVCGLDGKTYRNECALLKARCKEQPELEVQYQGRCKKTCRDVFCPGSSTCVVDQTNNAY
CVTCNRICPEPASSEQYLCGNDGVTYSSACHLRKATCLLGRSIGLAYEGKCIKAKSCEDIQCTG
GKKCLWDFKVGRGRCSLCDELCPDSKSDEPVCASDNATYASECAMKEAACSSGVLLEVKHS
GSCN (SEQ ID NO: 33)
A233 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSETRECIYYNANWELERTNQSGLERCEGDQDKRLHCYA
SWRNSSGTIELVKKGCWLDDINCYDRQECVATKENPQVYFCCCEGNFCNERFTHLPEAGGPE
VTYEPPPTAPT (SEQ ID NO: 34)
A234 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSETRECIYYNANWELERTNQSGLERCEGDQDKRLHCYA
SWRNSSGTIELVKKGCWLDDINCYDRQECVATKENPQVYFCCCEGNFCNERFTHLPEAGGPE
VTYEPPPTAPT (SEQ ID NO: 35)
A235 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSETRECIYYNANWELERTNQSGLERCEGEQDKRLHCYA
SWRNSSGTIELVKKGCWDDDFNCYDRQECVATEENPQVYFCCCEGNFCNERFTHLPEAGGP
EVTYEPPPT (SEQ ID NO: 36)
A236 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSGAILGRSETQECLFYNANWELERTNQTGVEPCEGEKD
KRLHCYATWRNISGSIEIVKKGCWLDDFNCYDRTDCVETEENPQVYFCCCEGNMCNEKFSYF
PEMEVTQPTS (SEQ ID NO: 37)
A239 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSTIPPHVQKSVNNDMIVTDNNGAVKFPQLCKFCDVRFST
CDNQKSCMSNCSITSICEKPQEVCVAVWRKNDENITLETVCHDPKLPYHDFILEDAASPKCIMK
EKKKPGETFFMCSCSSDECNDNIIFSEEYNTSNPD (SEQ ID NO: 38)
A240 Heavy chain amino acid sequence
EVQLLESGGGLVQPGGSLRLSCAASGFTFSSYAMSWVRQAPGKGLEWVSGITGSGGSTYYA
DSVKGRFTISRDNSKNTLYLQMNSLRAEDTAVYYCAKDPGTTVIMSWFDPWGQGTLVTVSSAS
TKGPSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAPSVFLFPPKPKDT
LMISRTPEVTCVVVDVSHEDPEVQFNWYVDGVEVHNAKTKPREEQFNSTFRVVSVLTVVHQD
WLNGKEYKCKVSNKGLPAPIEKTISKTKGQPREPQVYTLPPSREEMTKNQVSLTCLVKGFYPS
DIAVEWESNGQPENNYKTTPPMLDSDGSFFLYSKLTVDKSRWQQGNVFSCSVMHEALHNHYT
QKSLSLSPGKGGGSGGGGSGGGGSTIPPHVQKSDVEMEAQKDEIICPSCNRTAHPLRHINND
MIVTDNNGAVKFPQLCKFCDVRFSTCDNQKSCMSNCSITSICEKPQEVCVAVWRKNDENITLE
TVCHDPKLPYHDFILEDAASPKCIMKEKKKPGETFFMCSCSSDECNDNIIFSEEYNTSNPD
(SEQ ID NO: 39)
A242 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQDI
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSETQECL
FFNANWEKDRTNQTGVEPCYGDKDKRRHCFATWKNISGSIEIVKQGCWLDDINCYDRTDCVE
KKDSPEVYFCCCEGNMCNEKFSYFPEMEVTQPTSNPVTPKPP (SEQ ID NO: 40)
A243 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQDI
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSETRECIY
YNANWELERTNQSGLERCEGEQDKRLHCYASWRNSSGTIELVKKGCWLDDFNCYDRQECVA
TEENPQVYFCCCEGNFCNERFTHLPEAGGPEVTYEPPPTAPT (SEQ ID NO: 41)
A244 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQDI
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSGNCWLR
QAKNGRCQVLYKTELSKEECCSTGRLSTSWTEEDVNDNTLFKWMIFNGGAPNCIPCKETCEN
VDCGPGKKCRMNKKNKPRCVCAPDCSNITWKGPVCGLDGKTYRNECALLKARCKEQPELEV
QYQGRCKKTCRDVFCPGSSTCVVDQTNNAYCVTCNRICPEPASSEQYLCGNDGVTYSSACHL
RKATCLLGRSIGLAYEGKCIKAKSCEDIQCTGGKKCLWDFKVGRGRCSLCDELCPDSKSDEPV
CASDNATYASECAMKEAACSSGVLLEVKHSGSCNSISEDTEEEEEDEDQDYSFPISSILEW
(SEQ ID NO: 42)
A245 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQDI
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSGNCWLR
QAKNGRCQVLYKTELSKEECCSTGRLSTSWTEEDVNDNTLFKWMIFNGGAPNCIPCKETCEN
VDCGPGQSCVVDQTGSPRCVCAPDCSNITWKGPVCGLDGKTYRNECALLKARCKEQPELEV
QYQGRCKKTCRDVFCPGSSTCVVDQTNNAYCVTCNRICPEPASSEQYLCGNDGVTYSSACHL
RKATCLLGRSIGLAYEGKCIKAKSCEDIQCTGGKKCLWDFKVGRGRCSLCDELCPDSKSDEPV
CASDNATYASECAMKEAACSSGVLLEVKHSGSCNSISEDTEEEEEDEDQDYSFPISSILEW
(SEQ ID NO: 43)
A246 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQDI
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSGNCWLR
QAKNGRCQVLYKTELSKEECCSTGRLSTSWTEEDVNDNTLFKWMIFNGGAPNCIPCKETCEN
VDCGPGKKCRMNKKNKPRCVCAPDCSNITWKGPVCGLDGKTYRNECALLKARCKEQPELEV
QYQGRCKKTCRDVFCPGSSTCVVDQTNNAYCVTCNRICPEPASSEQYLCGNDGVTYSSACHL
RKATCLLGRSIGLAYEGKCIKAKSCEDIQCTGGKKCLWDFKVGRGRCSLCDELCPDSKSDEPV
CASDNATYASECAMKEAACSSGVLLEVKHSGSCN (SEQ ID NO: 44)
A247 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQDI
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSETRECIY
YNANWELERTNQSGLERCEGDQDKRLHCYASWRNSSGTIELVKKGCWLDDINCYDRQECVA
TKENPQVYFCCCEGNFCNERFTHLPEAGGPEVTYEPPPTAPT (SEQ ID NO: 45)
A248 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQDI
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSETRECIY
YNANWELERTNQSGLERCYGDKDKRRHCYASWRNSSGTIELVKKGCWLDDFNCYDRQECVA
TEENPQVYFCCCEGNFCNERFTHLPEAGGPEVTYEPPPTAPT (SEQ ID NO: 46)
A249 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQDI
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSETRECIY
YNANWELERTNQSGLERCEGEQDKRLHCYASWRNSSGTIELVKKGCWDDDFNCYDRQECVA
TEENPQVYFCCCEGNFCNERFTHLPEAGGPEVTYEPPPT (SEQ ID NO: 47)
A250 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQDI
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSGAILGRS
ETQECLFYNANWELERTNQTGVEPCEGEKDKRLHCYATWRNISGSIEIVKKGCWLDDFNCYD
RTDCVETEENPQVYFCCCEGNMCNEKFSYFPEMEVTQPTS (SEQ ID NO: 48)
A251 Heavy chain amino acid sequence
QVQLVQSGAEVKKPGASVKVSCKASGYTFTSYGLSWVRQAPGQGLEWMGWIIPYNGNTNSA
QKLQGRVTMTTDTSTSTAYMELRSLRSDDTAVYFCARDRDYGVNYDAFDIWGQGTMVTVSSA
STKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNVYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEALHN
HYTQKSLSLSPGKGGGSGGGGSGGGGSETFPPKYLHYDEETSHQLLCDKCPPGTYLKQHCT
AKWKTVCAPCPDHYYTDSWHTSDECLYCSPVCKELQYVKQECNRTHNRVCECKEGRYLEIEF
CLKHRSCPPGFGVVQAGTPERNTVCKRCPDGFFSNETSSKAPCRKHTNCSVFGLLLTQKGNA
THDNICSGNSESTQKCGIDVTLCEEAFFRFAVPTKFTPNWLSVLVDNLPGTKVNAESVERIKRQ
HSSQEQTFQLLKLWKHQNKDQDIVKKIIQDIDLCENSVQRHIGHANLTFEQLRSLMESLPGKKV
GAEDIEKTIKACKPSDQILKLLSLWRIKNGDQDTLKGLMHALKHSKTYHFPKTVTQSLKKTIRFLH
SFTMYKLYQKLFLEMIGNQVQSVKISCL (SEQ ID NO: 49)
A252 Heavy chain amino acid sequence
QVQLQESGPGLVKPSETLSLTCTVSGGSFSSHFWSWIRQPPGKGLEWIGYILYTGGTSFNPSL
KSRVSMSVGTSKNQFSLKLSSVTAADTAVYYCARARSGITFTGIIVPGSFDIWGQGTMVTVSSA
STKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNH
YTQKSLSLSLGKGGGSGGGGSGGGGSETFPPKYLHYDEETSHQLLCDKCPPGTYLKQHCTAK
WKTVCAPCPDHYYTDSWHTSDECLYCSPVCKELQYVKQECNRTHNRVCECKEGRYLEIEFCL
KHRSCPPGFGVVQAGTPERNTVCKRCPDGFFSNETSSKAPCRKHTNCSVFGLLLTQKGNATH
DNICSGNSESTQKCGIDVTLCEEAFFRFAVPTKFTPNWLSVLVDNLPGTKVNAESVERIKRQHS
SQEQTFQLLKLWKHQNKDQDIVKKIIQDIDLCENSVQRHIGHANLTFEQLRSLMESLPGKKVGA
EDIEKTIKACKPSDQILKLLSLWRIKNGDQDTLKGLMHALKHSKTYHFPKTVTQSLKKTIRFLHSF
TMYKLYQKLFLEMIGNQVQSVKISCL (SEQ ID NO: 50)
A253 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQD
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSTIPPHVG
KSVNNDMIVTDNNGAVKFPQLCKFCDVRFSTCDNQKSCMSNCSITSICEKPQEVCVAVWRKN
DENITLETVCHDPKLPYHDFILEDAASPKCIMKEKKKPGETFFMCSCSSDECNDNIIFSEEYNTS
NPD (SEQ ID NO: 51)
A254 amino acid sequence
ETFPPKYLHYDEETSHQLLCDKCPPGTYLKQHCTAKWKTVCAPCPDHYYTDSWHTSDECLYC
SPVCKELQYVKQECNRTHNRVCECKEGRYLEIEFCLKHRSCPPGFGVVQAGTPERNTVCKRC
PDGFFSNETSSKAPCRKHTNCSVFGLLLTQKGNATHDNICSGNSESTQKCGIDVTLCEEAFFRF
AVPTKFTPNWLSVLVDNLPGTKVNAESVERIKRQHSSQEQTFQLLKLWKHQNKDQDIVKKIIQD
DLCENSVQRHIGHANLTFEQLRSLMESLPGKKVGAEDIEKTIKACKPSDQILKLLSLWRIKNGDQ
DTLKGLMHALKHSKTYHFPKTVTQSLKKTIRFLHSFTMYKLYQKLFLEMIGNQVQSVKISCLEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGSGGGGSGGGGSTIPPHVQ
KSDVEMEAQKDEIICPSCNRTAHPLRHINNDMIVTDNNGAVKFPQLCKFCDVRFSTCDNQKSC
MSNCSITSICEKPQEVCVAVWRKNDENITLETVCHDPKLPYHDFILEDAASPKCIMKEKKKPGET
FFMCSCSSDECNDNIIFSEEYNTSNPD (SEQ ID NO: 52)
A255 Heavy chain amino acid sequence
QVQLVQSGAEVKKPGSSVKVSCKASGYTFSSNVISWVRQAPGQGLEWMGGVIPIVDIANYAQ
RFKGRVTITADESTSTTYMELSSLRSEDTAVYYCASTLGLVLDAMDYWGQGTLVTVSSASTKG
PSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPSCPAPEFLGGPSVFLFPPKPKDTL
MISRTPEVTCVVVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQ
KSLSLSLGKGGGSGGGGSGGGGSETFPPKYLHYDEETSHQLLCDKCPPGTYLKQHCTAKWK
TVCAPCPDHYYTDSWHTSDECLYCSPVCKELQYVKQECNRTHNRVCECKEGRYLEIEFCLKH
RSCPPGFGVVQAGTPERNTVCKRCPDGFFSNETSSKAPCRKHTNCSVFGLLLTQKGNATHDN
ICSGNSESTQKCGIDVTLCEEAFFRFAVPTKFTPNWLSVLVDNLPGTKVNAESVERIKRQHSSQ
EQTFQLLKLWKHQNKDQDIVKKIIQDIDLCENSVQRHIGHANLTFEQLRSLMESLPGKKVGAEDI
EKTIKACKPSDQILKLLSLWRIKNGDQDTLKGLMHALKHSKTYHFPKTVTQSLKKTIRFLHSFTM
YKLYQKLFLEMIGNQVQSVKISCL (SEQ ID NO: 53)
A237 Heavy chain amino acid sequence
QVQLVQSGAEVKKPGASVKVSCKASGYTFTSYGLSWVRQAPGQGLEWMGWIIPYNGNTNSA
QKLQGRVTMTTDTSTSTAYMELRSLRSDDTAVYFCARDRDYGVNYDAFDIWGQGTMVTVSSG
GGGSGGGGSGGGGSEVQLLESGGGLVQPGGSLRLSCAASGFTFSSYAMSWVRQAPGKGLE
WVSGITGSGGSTYYADSVKGRFTISRDNSKNTLYLQMNSLRAEDTAVYYCAKDPGTTVIMSWF
DPWGQGTLVTVSSASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTVERKCCVECPPCPAP
PVAGPSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSREEMT
KNQVSLTCLVKGFYPSDIAVEWESNGQPENNYKTTPPMLDSDGSFFLYSKLTVDKSRWQQGN
VFSCSVMHEALHNHYTQKSLSLSPGK (SEQ ID NO: 54)
A237 Light chain amino acid sequence
SYEVTQAPSVSVSPGQTASITCSGDKLGDKYACWYQQKPGQSPVLVIYQDSKRPSGIPERFSG
SNSGNTATLTISGTQAMDEADYYCQAWDSSTAVFGGGTKLTVLGGGGSGGGGSGGGGSEIV
LTQSPGTLSLSPGERATLSCRASQSVRGRYLAWYQQKPGQAPRLLIYGASSRATGIPDRFSGS
GSGTDFTLTISRLEPEDFAVFYCQQYGSSPRTFGQGTKVEIKRTVAAPSVFIFPPSDEQLKSGT
ASVVCLLNNFYPREAKVQWKVDNALQSGNSQESVTEQDSKDSTYSLSSTLTLSKADYEKHKVY
ACEVTHQGLSSPVTKSFNRGEC (SEQ ID NO: 55)
A238 Heavy chain amino acid sequence
QVQLQESGPGLVKPSETLSLTCTVSGGSFSSHFWSWIRQPPGKGLEWIGYILYTGGTSFNPSL
KSRVSMSVGTSKNQFSLKLSSVTAADTAVYYCARARSGITFTGIIVPGSFDIWGQGTMVTVSSG
GGGSGGGGSGGGGSEVQLLESGGGLVQPGGSLRLSCAASGFTFSSYAMSWVRQAPGKGLE
WVSGITGSGGSTYYADSVKGRFTISRDNSKNTLYLQMNSLRAEDTAVYYCAKDPGTTVIMSWF
DPWGQGTLVTVSSASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTVERKCCVECPPCPAP
PVAGPSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSREEMT
KNQVSLTCLVKGFYPSDIAVEWESNGQPENNYKTTPPMLDSDGSFFLYSKLTVDKSRWQQGN
VFSCSVMHEALHNHYTQKSLSLSPGK (SEQ ID NO: 56)
A238 Light chain amino acid sequence
EIVLTQSPGTLSLSPGERATLSCRASQSVSSSYLAWYQQKPGQAPRLLIYGASSRATGIPDRFS
GSGSGTDFTLTISRLEPEDFAVYYCQQYGSSPWTFGQGTKVEIKGGGGSGGGGSGGGGSEIV
LTQSPGTLSLSPGERATLSCRASQSVRGRYLAWYQQKPGQAPRLLIYGASSRATGIPDRFSGS
GSGTDFTLTISRLEPEDFAVFYCQQYGSSPRTFGQGTKVEIKRTVAAPSVFIFPPSDEQLKSGT
ASVVCLLNNFYPREAKVQWKVDNALQSGNSQESVTEQDSKDSTYSLSSTLTLSKADYEKHKVY
ACEVTHQGLSSPVTKSFNRGEC (SEQ ID NO: 57)
A241 Heavy chain amino acid sequence
QVQLVQSGAEVKKPGSSVKVSCKASGYTFSSNVISWVRQAPGQGLEWMGGVIPIVDIANYAQ
RFKGRVTITADESTSTTYMELSSLRSEDTAVYYCASTLGLVLDAMDYWGQGTLVTVSSGGGGS
GGGGSGGGGSEVQLLESGGGLVQPGGSLRLSCAASGFTFSSYAMSWVRQAPGKGLEWVSG
ITGSGGSTYYADSVKGRFTISRDNSKNTLYLQMNSLRAEDTAVYYCAKDPGTTVIMSWFDPWG
QGTLVTVSSASTKGPSVFPLAPCSRSTSESTAALGCLVKDYFPEPVTVSWNSGALTSGVHTFP
AVLQSSGLYSLSSVVTVPSSNFGTQTYTCNVDHKPSNTKVDKTVERKCCVECPPCPAPPVAGP
SVFLFPPKPKDTLMISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCS
VMHEALHNHYTQKSLSLSPGK (SEQ ID NO: 58)
A241 Light chain amino acid sequence
ETVLTQSPGTLSLSPGERATLSCRASQSLGSSYLAWYQQKPGQAPRLLIYGASSRAPGIPDRF
SGSGSGTDFTLTISRLEPEDFAVYYCQQYADSPITFGQGTRLEIKGGGGSGGGGSGGGGSEIV
LTQSPGTLSLSPGERATLSCRASQSVRGRYLAWYQQKPGQAPRLLIYGASSRATGIPDRFSGS
GSGTDFTLTISRLEPEDFAVFYCQQYGSSPRTFGQGTKVEIKRTVAAPSVFIFPPSDEQLKSGT
ASVVCLLNNFYPREAKVQWKVDNALQSGNSQESVTEQDSKDSTYSLSSTLTLSKADYEKHKVY
ACEVTHQGLSSPVTKSFNRGEC (SEQ ID NO: 59)
Peptide linker sequence GGGSGGGSGGGS (SEQ ID NO: 60)
Peptide linker sequence GGGS (SEQ ID NO: 61)
Peptide linker sequence GSSGGSGGSGGSG (SEQ ID NO: 62)
Peptide linker sequence GSSGT (SEQ ID NO: 63)
Peptide linker sequence GGGGSGGGGSGGGS (SEQ ID NO: 64)
Peptide linker sequence AEAAAKEAAAKEAAAKA (SEQ ID NO: 65)
Peptide linker sequence GGGGSGGGGSGGGGSGGGGS (SEQ ID NO: 66)
Peptide linker sequence GGGSGGGS (SEQ ID NO: 67)
Peptide linker sequence GSGST (SEQ ID NO: 68)
Peptide linker sequence GGSS (SEQ ID NO: 69)
Peptide linker sequence GGGGS (SEQ ID NO: 70)
Peptide linker sequence GGSG (SEQ ID NO: 71)
Peptide linker sequence SGGG (SEQ ID NO: 72)
Peptide linker sequence GSGS (SEQ ID NO: 73)
Peptide linker sequence GSGSGS (SEQ ID NO: 74)
Peptide linker sequence GSGSGSGS (SEQ ID NO: 75)
Peptide linker sequence GSGSGSGSGS (SEQ ID NO: 76)
Peptide linker sequence GSGSGSGSGSGS (SEQ ID NO: 77)
Peptide linker sequence GGGGGGGGS (SEQ ID NO: 78)
Peptide linker sequence GGGGSGGGGSGGGGS (SEQ ID NO: 79)

1-26. (canceled)

27. An isolated bifunctional antagonist molecule comprising a first antigen-binding molecule that specifically binds to RANKL, and a second antigen-binding molecule that specifically binds a ligand selected from the group consisting of Activin or Activin-related ligand or a TGF-β ligand, wherein the bifunctional antagonist molecule simultaneously neutralizes Activin- or TGF-β-mediated Smad signaling and RANKL-mediated NFκB signaling in a synergistic potent manner.

28. An isolated bifunctional antagonist molecule according to claim 27, wherein the first antigen-binding molecule that specifically binds to RANKL (“RANKL-binding polypeptide”) is selected from the group consisting of an anti-RANKL antibody or fragment of anti-RANKL antibody, wild-type osteoprotegerin (OPG) as well as modified OPG, and phage display-derived polypeptide capable of binding and sequestering RANKL; and wherein the second antigen-binding molecule that specifically binds to Activin or Activin-related ligand is selected from the group consisting of an anti-Activin antibody (including anti-Activin A antibody and anti-Activin B antibody), a fragment of anti-Activin antibody, wild-type Activin Type 2A Receptor (ActRIIA) or Activin Type 2B Receptor (ActRIIB) extracellular domains (ECDs), modified ActRIIA and ActRIIB extracellular domains, wild-type and modified native Activin-binding proteins such as follistatin, follistatin-like protein and pro-peptide, and a phage display-derived polypeptide targeting Activin or Activin-related ligand.

29. An isolated bifunctional antagonist molecule according to claim 27, wherein second antigen-binding molecule that specifically binds to Activin or Activin-related ligand is selected from the group of polypeptides comprising the amino acid sequence set forth in SEQ ID NOs: 1-17.

30. An isolated bifunctional antagonist molecule according to claim 27, wherein the first antigen-binding molecule that specifically binds to RANKL (“RANKL-binding polypeptide”) is selected from the group consisting of an anti-RANKL antibody or fragment of anti-RANKL antibody, wild-type osteoprotegerin (OPG) as well as modified OPG, and phage display-derived polypeptide capable of binding and sequestering RANKL; and wherein the second antigen-binding molecule that specifically binds to TGF-β ligand (“TGF-β-binding polypeptide”) is selected from the group consisting of an anti-TGF-β antibody, a fragment of anti-TGF-β antibody, wild-type TGF-β type-2 receptors (including TGFβRIIA and TGFβRIIB) extracellular domains (ECDs), modified TGFβRIIA and TGFβRIIB extracellular domains, and a phage display-derived antagonistic polypeptide targeting TGF-β ligand.

31. An isolated bifunctional antagonist molecule according to claim 30, wherein the second antigen-binding molecule that specifically binds to an TGF-β ligand selected from the group of polypeptides comprising the amino acid sequence set forth in SEQ ID NOs: 18-23.

32. An isolated bifunctional antagonist molecule according to claim 30, wherein the RANKL-binding molecule comprises an isolated anti-RANKL antibody, or antigen-binding fragment thereof, wherein the Activin-binding molecule comprises an isolated anti-Activin antibody, or antigen-binding fragment thereof, and wherein the TGF-β-binding molecule comprises an isolated anti-TGF-β antibody, or antigen-binding fragment thereof.

33. An isolated bifunctional antagonist molecule according to claim 32, wherein the isolated anti-RANKL antibody or antigen-binding fragment thereof and isolated anti-Activin antibody or antigen-binding fragment thereof and isolated anti-TGF-β antibody or antigen-binding fragment thereof is selected from the group consisting of monoclonal Abs (mAbs), polyclonal Abs, Ab fragments (e.g., Fab, Fab′, F(ab′)2, Fv, Fc, etc.), chimeric Abs, mini-Abs or domain Abs (dAbs), dual specific Abs, bispecific Abs, heteroconjugate Abs, single chain Abs (SCA), single chain variable region fragments (ScFv), humanized Abs, fully human Abs, and any other modified configuration of the immunoglobulin (Ig) molecule that comprises an antigen recognition site of the required specificity.

34. An isolated bifunctional antagonist molecule according to any one of claims 8 to 9, wherein the isolated antibody or antigen-binding fragment thereof is selected from the group consisting of a fully human, humanized and chimeric antibody.

35. An isolated bifunctional antagonist molecule according to claim 33, wherein the RANKL-binding molecule is an isolated antibody selected from the group consisting of an antibody comprising the heavy chain amino acid sequence set forth in SEQ ID NO: 24; an antibody comprising the light chain amino acid sequence set forth in SEQ ID NO: 25; an antibody comprising the heavy chain amino acid sequence set forth in SEQ ID NO: 24 and the light chain amino acid sequence set forth in SEQ ID NO: 25; an antibody comprising the heavy chain variable region amino acid sequence set forth in SEQ ID NO: 26; an antibody comprising the light chain variable region amino acid sequence set forth in SEQ ID NO: 27; and an antibody comprising the heavy chain variable region amino acid sequence set forth in SEQ ID NO: 26 and the light chain variable region amino acid sequence set forth in SEQ ID NO: 27; wherein the Activin-binding molecule is an isolated antibody selected from the group consisting of an antibody comprising the heavy chain amino acid sequence set forth in SEQ ID NO: 10; an antibody comprising the light chain amino acid sequence set forth in SEQ ID NO: 11; an antibody comprising the heavy chain amino acid sequence set forth in SEQ ID NO: 10 and the light chain amino acid sequence set forth in SEQ ID NO: 11; an antibody comprising the heavy chain variable region amino acid sequence set forth in SEQ ID NO: 12; an antibody comprising the light chain variable region amino acid sequence set forth in SEQ ID NO: 13; an antibody comprising the heavy chain variable region amino acid sequence set forth in SEQ ID NO: 12 and the light chain variable region amino acid sequence set forth in SEQ ID NO: 13; an antibody comprising the heavy chain amino acid sequence set forth in SEQ ID NO: 14; an antibody comprising the light chain amino acid sequence set forth in SEQ ID NO: 15; an antibody comprising the heavy chain amino acid sequence set forth in SEQ ID NO: 14 and the light chain amino acid sequence set forth in SEQ ID NO: 15; an antibody comprising the heavy chain variable region amino acid sequence set forth in SEQ ID NO: 16; an antibody comprising the light chain variable region amino acid sequence set forth in SEQ ID NO: 17; and an antibody comprising the heavy chain variable region amino acid sequence set forth in SEQ ID NO: 16 and the light chain variable region amino acid sequence set forth in SEQ ID NO: 17; and wherein the TGF-β-binding molecule is an isolated antibody selected from the group consisting of an antibody comprising the heavy chain amino acid sequence set forth in SEQ ID NO: 20; an antibody comprising the light chain amino acid sequence set forth in SEQ ID NO: 21; an antibody comprising the heavy chain variable region amino acid sequence set forth in SEQ ID NO: 22; an antibody comprising the light chain variable region amino acid sequence set forth in SEQ ID NO: 23; and an antibody comprising the heavy chain variable region amino acid sequence set forth in SEQ ID NO: 22 and the light chain variable region amino acid sequence set forth in SEQ ID NO: 23.

36. An isolated bifunctional antagonist molecule according to claim 27, wherein the isolated bifunctional antagonist molecule comprises an amino acid sequence selected from the group consisting of the amino acid sequences set forth in SEQ ID NOs: 40-48 and 51-52.

37. An isolated bifunctional antagonist molecule according to claim 27, wherein the isolated bifunctional antagonist molecule comprises a heavy chain selected from the group consisting of a heavy chain comprising the amino acid sequence set forth in SEQ ID NOs: 29-39 and a light chain comprising the amino acid sequence set forth in SEQ ID NO: 25.

38. An isolated bifunctional antagonist molecule according to claim 27, wherein the isolated bifunctional antagonist molecule is selected from the group consisting of: an isolated bifunctional antagonist molecule comprising the heavy chain amino acid sequence set forth in SEQ ID NO: 49 and the light chain amino acid sequence set forth in SEQ ID NO: 11; an isolated bifunctional antagonist molecule comprising the heavy chain amino acid sequence set forth in SEQ ID NO: 50 and the light chain amino acid sequence set forth in SEQ ID NO: 15; and an isolated bifunctional antagonist molecule comprising the heavy chain amino acid sequence set forth in SEQ ID NO: 53 and the light chain amino acid sequence set forth in SEQ ID NO: 21.

39. An isolated bifunctional antagonist molecule according to claim 27, wherein the isolated bifunctional antagonist molecule is selected from the group consisting of: an isolated bifunctional antagonist molecule comprising the heavy chain amino acid sequence set forth in SEQ ID NO: 24, the light chain amino acid sequence set forth in SEQ ID NO: 25, the heavy chain amino acid sequence set forth in SEQ ID NO: 10 and the light chain amino acid sequence set forth in SEQ ID NO: 11; an isolated bifunctional antagonist molecule comprising the heavy chain amino acid sequence set forth in SEQ ID NO: 24, the light chain amino acid sequence set forth in SEQ ID NO: 25, the heavy chain amino acid sequence set forth in SEQ ID NO: 14 and the light chain amino acid sequence set forth in SEQ ID NO: 15; an isolated bifunctional antagonist molecule comprising the heavy chain amino acid sequence set forth in SEQ ID NO: 24, the light chain amino acid sequence set forth in SEQ ID NO: 25, the heavy chain amino acid sequence set forth in SEQ ID NO: 20 and the light chain amino acid sequence set forth in SEQ ID NO: 21.

40. A pharmaceutical composition comprising a therapeutically effective amount of the isolated bifunctional antagonist molecule according to claim 27 in admixture with a pharmaceutically acceptable carrier.

41. A method of treating or preventing a disease condition whose pathogenesis involves the activation of both RANKL-NFκB and Activin/TGFβ-Smad2/3 signaling pathways, comprising administering to said subject a therapeutically effective amount of the composition of claim 40 to the subject.

42. A method according to claim 19, wherein the disease condition is a cancer selected from the group consisting of melanoma, multiple myeloma, lung cancer, pancreatic cancer, colorectal cancer, liver cancer, gastric cancer, kidney cancer, bladder cancer, head and neck cancer, thyroid cancer, breast cancer, ovarian cancer, endometrial cancer, testicular cancer, prostate cancer and brain cancer.

43. A method according to claim 42, wherein the method further comprises co-therapy in combination with and immune checkpoint inhibitor selected from the group consisting of an anti-PD1, anti-PDL1, and anti-CTL4 antibodies.

44. An isolated nucleic acid molecule comprising a polynucleotide encoding a bifunctional antagonist molecule according to claim 27.

45. A recombinant vector comprising the nucleic acid molecule of claim 44.

46. A host cell comprising the recombinant vector of claim 45.
